(19) United States
(12) Patent Application Publication
(10) Pub. No.: US 2008/0070971 A1
(43) Pub. Date: Mar. 20, 2008

(54) Medical Use of Bilirubin and its Structural Analogues

(76) Inventor: Xiang H. Wang, Alpharetta, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 11/682,797

(22) Filed: Mar. 6, 2007

Related U.S. Application Data
(60) Provisional application No. 60/779,480, filed on Mar. 6, 2006.
(60) Provisional application No. 60/779,653, filed on Mar. 6, 2006.

(51) Int. Cl. A61K 31/4025; A61K 31/045

(52) U.S. Cl. 514/422; 514/423; 514/424; 514/730

(57) ABSTRACT

Formulations and methods for preventing, inhibiting or controlling metabolic disorder, age-related disease and acute inflammations have been developed. The compositions comprise of bilirubins, bilirubin derivatives, their tetrapyrrolic analogues, tripyrroles, and dipyrroles. The compositions can be administered as a dosage form for oral ingestion, injection, suppository, or topical application. The effective amount of the compound is typically from 0.001-100 mg/kg body weight, preferably in the range from 0.01-50 mg/kg body weight, and most preferably from 0.05-10 mg/kg body weight. Examples demonstrate the efficacy of the compounds in both in vitro and in vivo tests.

### DROSS-REFERENCE TO RELATED APPLICATIONS

This application claims priority to U.S. Ser. No. 60/779,480 filed on Mar. 6, 2006 and U.S. Ser. No. 60,779,653 filed on Mar. 6, 2006. The disclosures in the applications listed above are herein incorporated by reference.

### FIELD OF THE INVENTION

The present invention is generally in the field of pharmaceutical compositions comprising a bilirubin or linear tetrapyrrole, tripyrrole, or dipyrrole analogue of bilirubin for preventing, controlling and treating metabolic disorders.

### BACKGROUND OF THE INVENTION

Bilirubin is a breakdown product of normal heme catabolism. During the metabolic process, erythrocytes (red blood cells) are destroyed when they get old or damaged, releasing hemoglobin. Hemoglobin is broken down to heme, which is converted into biliverdin by heme oxygenase and then further reduced to bilirubin by biliverdin reductase. Bilirubin is then bound to albumin and sent to the liver where it is conjugated with glucuronic acid by bilirubin-UDP glucuronosyltransferase (UDPGT) to bilirubin mono- and di-glucuronides, making it water soluble. The bilirubin-glucuronides are delivered from bile into the intestine wherein they are further transformed to urobilin, urobilinogen, stereobilin and stereobilinogen by successive reductions. Bilirubin glucuronides can be de-conjugated by glucuronidase in the intestine, which allows bilirubin to be reabsorbed and recycled back to the liver and circulation system.

Structurally, bilirubin consists of an open chain of four pyrroles, i.e., linear tetrapyrrole; by contrast, heme, the parent molecule of bilirubin, has a closed ring of four pyrroles, called protoporphyrin. Heme and other porphyrins have been studied extensively for medical applications. For example, heme (lyophilized hematin for injection) has been approved by the U.S. Food and Drug Administration for the amelioration of recurrent attacks of acute intermittent porphyria temporally related to the menstrual cycle in susceptible women. The metal complexes of porphyrins are inhibitors of many enzymes of physiological importance and are candidates for the pharmaceutical use (Louie and Meade, Chem, Rev., 1999, 99:2711-2734). U.S. Pat. Nos. 5,430,051 and 5,567,409 described the use of porphyrins having at least one carboxylic group for diagnosis and treatment of rheumatoid arthritis in combination with phototherapy. U.S. Pat. Nos. 4,656,186; 4,691,885; 4,977,177; 4,675,338; 5,004,811; 6,479,477; 6,544,975; 6,583,132 and 6,916,799 described the use of porphyrins and their metal-complexes for scavenging oxidative species and modulating the intra- or extracellular levels of oxidants. European Patent EP 0,782,455 presented a method of using porphyrin-based compounds for treatment of multiple sclerosis. U.S. Pat. No. 5,948,771 showed a method for using metal-porphyrins as an inhibitor for guanylyl cyclase in cardiac myocytes to improve cardiac health. U.S. Pat. No. 5,756,492 described the use of metalloporphyrrins in transplantation to enhance the survival of the organ. U.S. Pat. NO. 5,629,198 described the use of metal porphyrins for treating HIV infection. U.S. Pat. No. 6,951,640 described the application of metal-porphyrins in radiation treatment of tumors.

On contrary, medical applications of bilirubin and its structural analogues of linear tetrapyrroles have received little attention. In the Chinese tradition medicine, gallbladder from ox (called niuhuang, or Calculus Bovis) and other bile liquid from animals (such as snake and bear) have been used as sedative, anticonvulsant and bacteriostatic materials to treat symptoms of delirium, convulsions, loss of consciousness in feverish diseases, swellings in the throat and mouth, and phlegm disorders. The major component of Niuhuang is bile acid (ursodeoxycholic acids), together with other minor ingredients including bilirubin and other bile pigments. Natural Niuhuang generally contains high content of heavy metals including cadmium, lead and arsenic oxides which could be responsible for patient intoxication in using (Deng, Zhongyao Tongbao, 1993, 8:19-20; Dong et al, Guangpuxue Yu Guangpufenxi, 1999, 19:417-418; Wang, Zhaongguo Zhongxiyi Jiehezhai, 2005, 35:213). Despite these possible adverse effects, in the Chinese medicine practice, synthetic niuhuang, and so bilirubin, is considered to be less effective or potent in therapeutic effect, and natural ox bile stones are preferred and always used in combination with other medicinally active compounds. Many niuhuang-branded drugs sold in China contain no niuhuang or bilirubin at all.

Several recent studies have noted possible health benefits of bilirubin. Stocker and colleagues reported that bilirubin and its conjugates are powerful antioxidants that prevent the peroxidation of LDL by scavenging the free oxidative radicals (Stocker et al, Proc. NAS, 1987, 84:5918-5922; Stocker and Ames, 1987; Frei et al, Proc. NAS, 1988, 85:9748-9752; Neuzil and Stocker, J. Biol. Chem., 1994, 269:16712-16719; Stocker, Antioxidants & Redox Signaling, 2004, 6:841). U.S. Patent Application Publication Nos. US2003/0069281 and US2004/0039211 described the use of metal complexes (zinc, iron, nickel, cobalt, and manganese) of biliverdin esters as antioxidants for modulating the cellular levels of oxidants. U.S. Pat. No. 5,380,667 by Schwertner describes an inverse relationship between the risk of cardio vascular disease and total serum bilirubin. A patient would be at increased risk for severe CHD when the serum bilirubin level was below the threshold bilirubin level of 0.6 mg/dl. Thereafter, several studies have reported similar findings for people of different ethnical groups, locations, sex, and age groups (Morento et al, Clin. Chem., 1994, 1791-1792; Breimer et al, Clinc. Chem., 1995, 91:489-492; Hopkins, Artherosclerosis, Thrombosis, and Vascular Biology, 1996, 16:250-255; Hunt et al, 1996, Levinson, am. Clin. Lab. Sci., 1997, 27:185-192; Madhaven et al, 1997; Schwertner, Artherosclerosis, 1998, 136:383-387; Olineseu et al, Rom. J. Intern. Med., 1999, 37;239-249; Padadkis et al, Am. J. Hypertens., 1999, 12:673-681; Sun, et al, J. Clin. Diag., 1999, 17:1-3; U.S. Pat. No. 5,380,667; Schwertner and Fisher, Atheroselerosis, 2000, 150:381-387; U.S. Pat. No. 6,869,802 to Schertner; Mayer, Clin. Chem., 2000, 46:1723-1727, Ishizaka et al, Stroke, 2001, 32:580-581; Djousse et al, Circulation, 2002, 106:2919-2924;Lin et al, J. Clin. Cardiol., 2002, 18:204-206; Vitek et al, Artherosclerosis, 2002, 160:449-456; Bosma et al, Clinc. Chem., 2003, 49:1180-1181; Endler et al, Clinc. Chem., 2003, 49:1201-1204; Hu et al, J. Clin. Cardiol., 2004, 20:180-181; Wiedemann et al, NY State J. Med., 2004, 91:493-496; Gullu et al, Artherosclerosis, Thromb. Vase. Biol., 2005, 25;1-6; Pan et al, Chinese J. Clin. Med., 2005, 4:12-14). U.S. Pat. Nos. 6,720,489 and 6,905,880 also reported that serum bilirubin was a risk factor for predicting cancer mortality, rheumatoid arthritis, and all-cause mortality. Recently, it was reported that lower serum bilirubin levels appeared to have an increased risk of colorectal cancer (Zucker et al, Hepatol., 2004, 40:827-835).

No attempts have been made to use bilirubin for medial applications. Bilirubin has always been considered highly toxic to human (e.g., Hansen, Clin, Perionatol., 2002, 29:765-778; Wennberg, N.Y. State J. Med., 1991, 91:493-496; Bratlid, NY State J. Med., 1991, 91:489-492). Elevated levels of serum bilirubin, a phenomenon called hyperbilirubinemia or jaundice, are believed to be pathogenic, cause kemicterus and seizure, and damage brain and other organs. Much research effort has been directed to developing medications to eliminate bilirubin from human body in order to reduce the serum bilirubin level (e.g., U.S. Pat. Nos. ,3658,068; 4,770,997; 4,996,200; 4,985,360; and 5,624,811; and U.S. Patent Applications 200440242509 and 200600100675; european Patent Documents Nos. EP0140004; EP0247847; and EP0320095). For example, neonatal hyperbilirubinemia is usually treated by phototherapy before the infant is discharged from hospital. In another example, the elevation of serum bilirubin level in AIDS patients in association with the use of anti-HIV drugs such as Atazanavir and Indinavir has been considered as a serious side effect, and additional drugs aimed at avoiding the serum bilirubin elevation have been recommended (McPhee et al, Biochem. J., 1996, 320:681-686; Mori et al, Jpn. J. Cancer Res., 1991, 82:755-757). These examples of medical practices demonstrate the widespread conception that bilirubin is toxic and that elevated serum bilirubin level causes serious adverse health effects and should be treated.

It is an object of the present invention to prevent, control and/or treat cardiovascular diseases including atheroselerosis and stroke through the application of formulations to reduce the blood levels of total cholesterol, triglyceride and low density lipoprotein (LDL).

### SUMMARY OF THE INVENTION

Methods and compositions for preventing, controlling or treating a metabolic disorder such as a high blood cholesterol, overweight and obesity, aging-related diseases including cardiovascular disease, rheumatoid arthritis, cancer and Alzheimer's disease, and acute inflammatory conditions like allergy, asthma, and sunburn have been developed. The formulations comprise a bilirubin or linear tetrapyrrole, tripyrrole, or dipyrrole analogue of bilirubin. The methods of treatment include:

increasing total serum bilirubin level through (i) direct replenishing bilirubin, or (ii) administrating bilirubin pre-drugs, including heme, hematin, hemin, and its protoporphyrin analogues, which generate bilirubin via metabolic transformation, or (iii) supplying derivatives and analogues of bilirubin, or (iv) reducing bilirubin excretion with bilirubin-UDPGT enzyme inhibitors, or (v) a combination of (i), (ii), (iii) and (iv).

reducing the blood levels of total cholesterol, triglyceride and low density lipoprotein (LDL) cholesterols through the mechanisms consisting of

(i) inhibiting the activity of pancreatic lipases, phospholipases, and/or cholesterol esterases to minimize the fatty food absorption;

(ii) inhibiting the activity of cholesterol and fat synthesis enzymes selected from HMG-CoA reductases, glycerol acyl esterase, isocitrate dehydrogenases, malic dehydrogenases, and glucose-6-phosphate dehydrogenase;

(iii) a combination of both (i) and (ii);

minimizing or preventing acute or chronic inflammations through (i) scavenging reactive free radicals; (ii) suppressing generation of inflammatory mediators by inhibiting the activity of the enzymes initiate and control the inflammatory reactions, including but not limited to phospholipase A2, cyclooxygenase, and lipoxygenase; (iii) a combination of both (i) and (ii); regulating the activity, amount, or both amount and activity of enzymes that control the cellular processes of cancer and aging, which include but not limited to histone deacetylase and acetylcholinesterase.

The effective amount of the compositions is typically from 0.001-100 mg/kg body weight, preferably in the range from 0.01-50 mg/kg body weight, more preferably from 0.05-25 mg/kg body weight, and most preferably from 0.1-10 mg/kg body weight. The formulations can be administered as a dosage form for oral ingestion, injection, suppository, or topical application.

One specific treatment target is to increase the level of total serum bilirubin (TSB) while simultaneously lower the level of total cholesterol (TC) to maintain an optimal ratio of TC/TSB, which is generally in the range of 100 to 400 when their concentration are expressed in in mmol/L, or 60 to 250 when compared in mg/dL, but preferably between 100 and 300 when compared in mmol/L, or 60 to 180 when compared in mg/dL.

Examples demonstrate the efficacy of these compounds in both in vivo and in vitro tests.

### BRIEF DESCRIPTION OF THE DRAWINGS

FIG. 1 is a graph of the percent inhibition of phospholipase A2 (PLA2) and cholesterol esterase (CHE) as a function of bilirubin concentration at pH=7.2.

FIG. 2 is a graph of the percent inhibition of acetylcholinesterase activities as a function of bilirubin concentration.

FIG. 3 is a graph of the percent inhibition of cyclooxygenase (COX) enzyme activities as a function of bilirubin concentration.

FIG. 4 is a graph comparing COX inhibition by bilirubin and hemin.

FIG. 5 is a graph of the inhibition of 3-hydroxy-3-methylglutaryl-Coenzyme A (HMG-CoA) reductase as a function of bilirubin concentration.

FIG. 6 is a graph comparing inhibition of HMG-CoA reductase by bilirubin, biliverdin and hemin at a dosage of 30 mg/L.

FIG. 7 si a graph of the inhibition of isocitrate dehydrogenase (ICDH) and malate dehydrogenase (MDH) as a function of bilirubin concentration.

FIG. 8 is a graph of the minimum ethryma dose (MED), as measured by the time of irradiation to induce ethryma, as a function of dosage of bilirubin treatment.

FIG. 9 is a graph of the inhibition of histone deacetylase as a function of bilirubin concentration.

FIG. 10 is graph of the relative cancer cell growth as a function of bilirubin concentration.

FIG. 11 is a graph of total serum bilirubin (TSB), total cholesterol (TC) and triglyceride (TG) with and without bilirubin supplementation.

FIG. 12 is a graph of the total cholesterol (TC) as a function of total serum bilirubin (TSB).

FIG. 13 is a graph of the distribution (%) of people as a function of TC/TSB, the ratio of the total cholesterol over total serum bilirubin, for healthy people and cardiovascular disease patients.

### DETAILED DESCRIPTION OF THE INVENTION

### Definitions

The definitions and their meanings of the various terms used herein are given as follows.

Bilirubin pre-drugs: substances that can lead to formation of bilirubin through metabolic process, including but not limited, to heme, hematin, hemoglobins, and cytochromes. They are also called bilirubin precursors.

Bilirubin derivatives: substances that are intermediate or final products of bilirubin metabolic transformation (i.e., oxidation or reduction ), including biliverdin, urobilin, urobilinogen, stereobilin, urobiliverdin, bilirhodia, biliviolin, mesobiliviolin, phycobiliviolin, and stereobilinogen.

Analogues of bilirubin and derivatives: linear tetrapyrroles that have the backbone of bilirubin or its derivatives.

Functional groups R1, R2, . . . , and R11 (in linear tetrapyrrole, tripyrrole and depyrrole structure formulas): radical groups selected from the following formula:
hydrogen: —H;
oxygen: ═O;
hydroxyl: —OH;
straight chain or branched alkanes: —(CH₂)ₙCH₃;
straight chain or branched alkenes: —(CH₂)ₙCH═CH(CH ₂)ₘCH₃;
saturated straight chain or branched aliphatic fatty acids: —(CH₂)ₙCO₂H;
unsaturated straight chain or branched fatty acids: —(CH₂)ₙCH═CH(CH₂)ₘCO₂H;
straight or branched aliphatic fatty esters: —(CH₂)ₙCH₂OH, —(CH₂)ₙCH═CH(CH₂)ₘCH₂OH;
alkoxyls: —O(CH₂)ₙCH₃;
alkylsulfonic acids: —(CH₂)ₙSO₃H, or —(CH₂)ₙCH═CH(CH₂)ₘSO₃H;
alkylsulfate: —(CH₂)ₙOSO₃H, or —(CH₂)ₙCH═CH(CH₂)ₘOSO₃H;

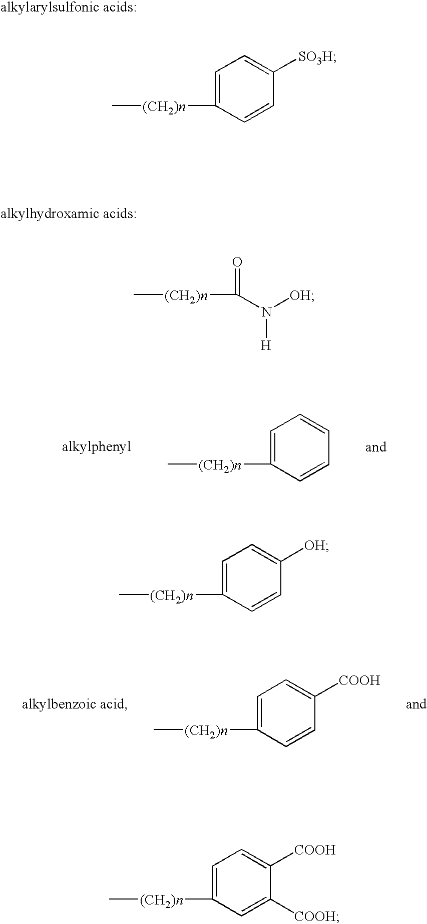

wherein the hydrocarbon chain length (n and m) ranges from 0 to 20.

Metabolic disorder: a health condition characterized by one or more of the following syndromes: high blood cholesterol, high blood triglycerides, overweight, and obesity.

Age-related diseases: diseases associated with the aging process which risk order increases with age, including cardiovascular disease, rheumatoid arthritis, Alzheimer's disease, and cancer.

Inflammatory diseases: diseases caused by or exacerbated in acute and/or chronic inflammatory reactions, including but not limited to atheroselerosis, asthma, arthritis, sunburn and allergies.

UDPGT: uridine diphosphate glucuronsyltransferase.

HMG-CoA: hydroxymethylglutaryl-Coenzyme A.

NAD: nicotinamide adenine dinucleatide; NADH: reduced form of NAD.

NADPH: nicotinamide adenine dinucleatide phosphate; NADPH₂: reduced from of NADPH.

### I. Compositions

A. Compounds

### Linear Tetrapyrroles

The general structure of the linear tetrapyrroles is depicted by the following formula

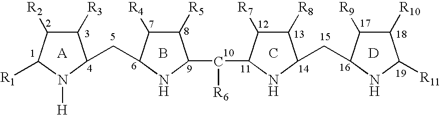

where the four pyrroles (labeled as A, B, C and D) are linked by either methine (—CH═) or methylene (—CH₂═) groups, forming the structural

backbone which carbons are numbered from 1 to 19; and the functional

groups R1, . . . R11 are given in the Definition.

### Among linear tetrapyrroles, 1,19-bilindiones of the following general formula,

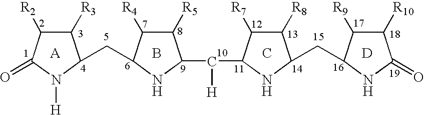

are the pigements widely distributed in nature. Depending on the linkages between the pyrroles, a broad range of subfamilies of linear tetrapyrroles can be obtained. The following groups are some examples.
(a) Biliverdins

In biliverdins, the four pyrroles are inked by three methine groups (where R6=hydrogen), as shown below:

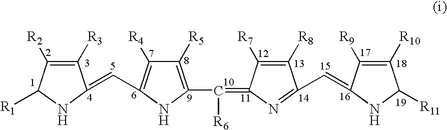

where the functional groups are described in the Definition.

Mammals, biliverdins are produced from the break down of heme which has a closed (i.e., ring) tetrapyrrole structure. Depending on the breaking position in ring structure, α, β, γ, and δ biliverdins can be obtained, which have the general 1,19-bilindione structure as shown below:

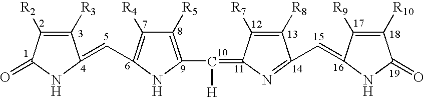

The biliverdin commonly found in mammals is α-biliverdin, i.e., biliverdin IXα, represented by the following formula:

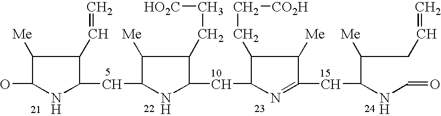

Its systematic name is 2,7,13,17-tetramethyl-1,19-dioxo-3,18-divinyl-1,19,22,24-tetrahydro-21H-biline-8,12-dipropanoic acid. The vinyl groups in the above formula can undergo hydrogenation to form mesobiliverdin having the following formula:

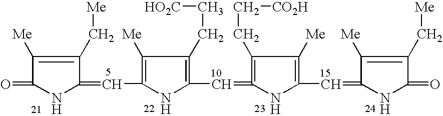

### Its systematic name is 3,18-diethyl-2,7,13,17-tetramethyl-1,19-dioxo-1,19,22,24-tetrahydro-21H-biline-8,12-dipropanoic acid.

(b) Bilirubins

In bilirubins, the central linkage for the dipyrroles is methylene (when R6=hydrogen), while the two terminal pyrroles are linked by methine groups, as shown below

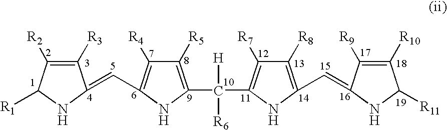

where the functional groups are given in Definition.

In vivo, bilirubins are derived from biliverdins by biliverdin reductase and are of 1,19-bilindione structure, as shown below:

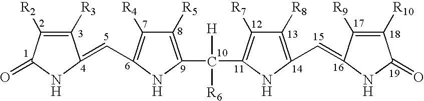

Like biliverdins, bilirubins can exist in α, β, γ, and δ structures. The most commonly encountered form is bilirubin IXα, in which the functional groups R5 and R7 are propionic acids, R3 and R10 are vinyl groups, and R2, R4, R8 and R9 are methyl groups, as shown below

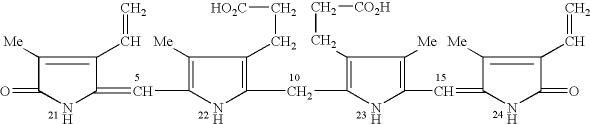

The systematic name is: 2,7,13,17-tetramethyl-1,19-dioxo-3,18-divinyl-1,10,19,22,23,24-hexahydro-21H-biline-8,12-dipropanoic acid. When the vinyl groups are reduced to ethylene groups through hydrogenation, the bilirubin is transformed into its isomer, mesobilirubin, having the following formula:

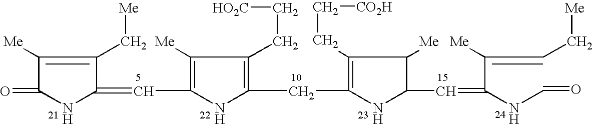

Its systematic name is: 3,18-diethyl-2,7,13,17-tetramethyl-1,19-dioxo-1,19,22,24-tetrahydro-21H-biline-8,12-dipropanoic acid.

Bilirubin can be conjugated or bound. In the plasma, bilirubin is usually bound to albumin and then transported to the liver, where it is conjugated with glucuronic acid to form bilirubin mono- and di-glucuronide by the microsomal enzyme UDP-glucuronyltransferase, rendering bilirubin water-soluble. The bilirubin conjugates are excreted from the bile into the intestine. Further metabolic transformations of bilirubin consist in a chain of reductions through the conversion of the two terminal methine groups (—CH═) to methylene groups (—CH═) and through the complementary partial hydrogenation of the two terminal pyrrole rings to produce a wide range of metabolites, including urobilin, stereobilinogen, stercobilin, mesobiliviolin, mesobiliviolin, and half-stereobilin, and urobilinogen. Their structures are described below.

(c) Urobilin (Urobilin IXα)

Urobilin is a metabolic product of bilirubin found in human urine. Its structural formula is:

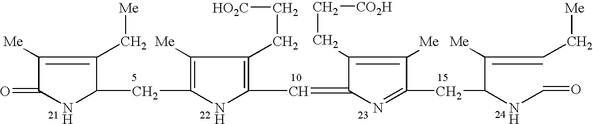

Its systematic name is 3,18-diethyl-2,7,13,17-tetramethyl-1,19-dioxo-1,4,5,15,16,19,22,24-octahydro-21H-biline-8,12-dipropanoic acid. The general structural formula of urobilin analogues is:

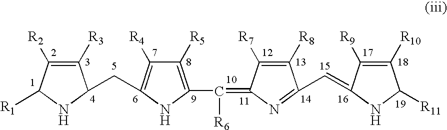

(d) Urobilinogen (Urobilinogen IXα, Mesobilirubinogen)

Urobilinogen is usually the final metabolic product of bilirubin transformation found in urine, in which all the methine groups are reduced to methylene groups, as shown below:

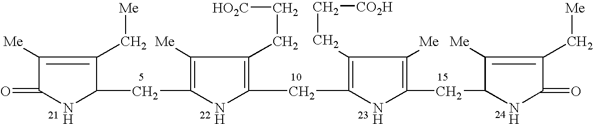

Its systematic name is 2,17-diethyl-3,7,13,18-tetramethyl-1,19-dioxo-1,4,5,10,15,16,19,22,23,24-decahydro-21H-biline-8,12-dipropanoic acid. The general structural formula of urobilinogen analogue is represented by

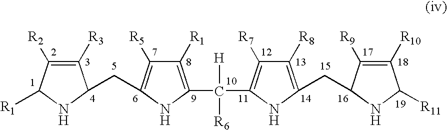

(e) Stercobilin (Stercobilin IXα)

Stercobilin is the bilirubin metabolic product usually found in feces. Hydrogenation of the two terminal pyrroles are completed in stercobilin, but the center methine group is unchanged:

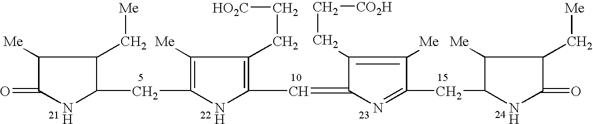

Its systematic name is 3,18-diethyl2,7,13,17-tetramethyl-1,19-dioxo-1,2,3,4,5,15,16,17,18,19,22,24-dodecahydro-21H-biline-8,12-dipropanoic acid. The general structure formulation of stercobilin analogues is:

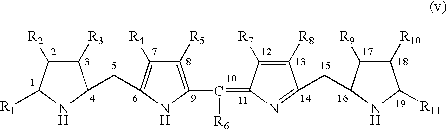

where the functional groups are given in the Definition.

### The structures of the analogues of other bilirubin derivatives are represented by formulas (vi)-(x).

(f) Phycobiliverdins

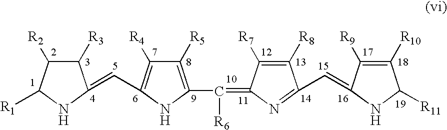

(g) Stereobilinogens

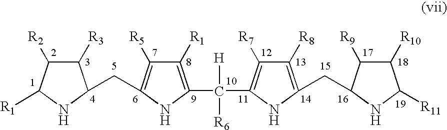

(h) Mesobiliviolin

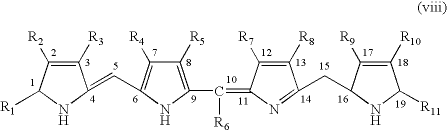

(i) Half-stereobilin

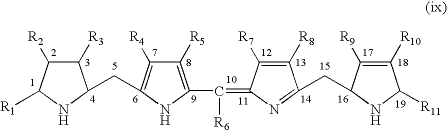

(j) Mesobilirhodin (or phycobiliviolin)

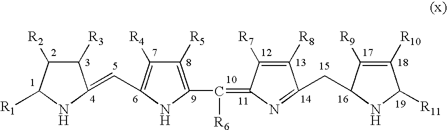

### Tripyrroles and Dipyrroles

Tripyrroles and dipyrroles are relatives of linear tetrapyrroles. Dipyrroles are sometimes called half-bilirubin or semi-rubin. These compounds have many similar properties to the linear tetrapyrroles. For example, they are potent antioxidants and inhibitors for many enzymes. The general structures of tripyrroles and dipyrroles are described as follows.

The general structural formula of tripyrroles is:

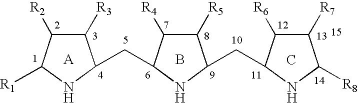

The 1,14-dione form of tripyrroles have the following structure.

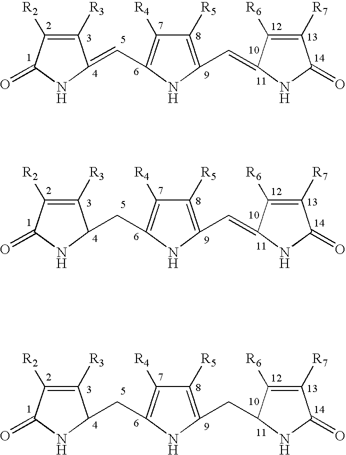

The general structure formula of dipyrroles is described as follows

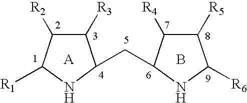

The dipyrrinones have the following formulas:

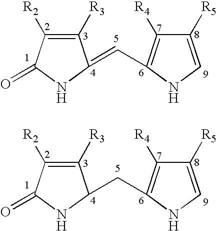

where the functional groups are given in the Definition.

An example of tripyrroles is uroerythrin which has been separated and identified form human urine. It has following structure formula:

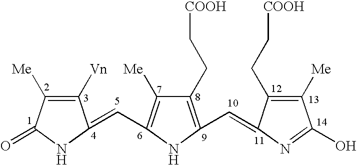

where Me=methylene and Vn=vinyl. Its systematic name is 1,14,15,17-tetrahydro-2,7,13-trimethyl-1,14-dioxo-3-vinyl-16H-tripyrrin-8,12-dipropionic acid.

An example of dipyrroles is semi-bilirubin, having the following structure:

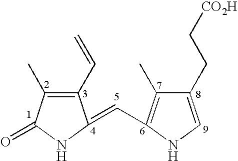

Methods for producing the various compounds of linear tetrapyrroles, tripyrroles and dipyrroles can be found in the literature. For example, the synthesis of aliphatic fatty acid bilirubins (in which the propionic acids at C8 and C12 are replaced by aliphatic fatty acids with a hydrocarbon chain length ranging from 3 to 30), benzoic bilirubins (in which the propionic acids are replaced by benzoic acids), and sulfonic bilirubin (in which the propionic acids are replaced by sulfonic acids) has been reported (Thyrann and Lightner, Tetrahedron, 1996,52:447-460; Boiadjiev and Lightner, Monatschefte fur Cheie, 2001, 132:1201-1212; Boiadjiev & Lightner, J. Org. Chem., 2003, 68:7591-7604). Synthesis of dipyrroles was described by Chen et al (2002). Table 1 gives some examples of bilirubins and their structural analogues, while Table 2 shows examples of biliverdins and their structural analogues.

The compounds of the above bilirubins, derivatives of bilirubins, analogues of biliruins and their derivatives, tripyrroles and dipyrroles can be used either as the compounds by themselves, or as complexes with metals consisting of sodium, potassium, calcium, magnesium, manganese, iron, zinc and copper, or as conjugates consisting of glucuronides, taurates, albumins, and amine acids, or in combination.

Heme and Its Analogues

Heme is the parent molecule of bilirubin. The metabolism of hemoglobin generates heme, an iron-protoporphyrin IX complex, which is converted into biliverdin by heme oxygenase and then biliverdin is further reduced to bilirubin by biliverdin reductase. Each mode of heme generates one mole of bilirubin. Therefore, heme and its analogues (protoporphyrrins) of the following formula are the pre-drugs of bilirubins.

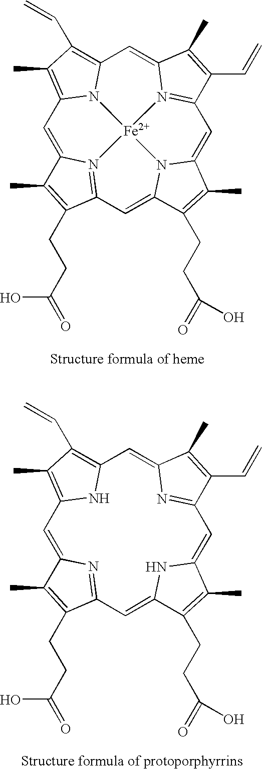

The oxidation state of the iron ion and the identity of the negatively charged counter-ion determine the name of the product. If iron (II) is present the product is heme. If iron (III) is present and the counterion is chloride, the product is called hemin. If iron (III) is present and the counterion is hydroxide, then it is called hematin. Heme (lyophilized hematin for injection) has been approved by the U.S. Food and Drug Administration for the amelioration of recurrent attacks of acute intermittent porphyria temporally related to the menstrual cycle in susceptible women. Since heme is more stable when bound to proteins or amino acids such as arginine and lysine, the heme derivatives, i.e., the heme-protein or heme-amino acid complexes, have been used to replace heme outside the U.S. The heme derivatives are reported to have the same therapeutic effects as heme.

Flavonoids

Flavonoids include bioflavonoids, derived from 2-phenylchromone (2-phenyl-1,4-benzopyrone) structure, isoflavonoids, derived from the 3-phenylchromone (3-phenyl-1,4-benzopyrone) structure, and neoflavonoids, derived from the 4-phenylcoumarine (4-phenyl-1,2-benzopyrone) structure. The structure of flavanoids is represented by

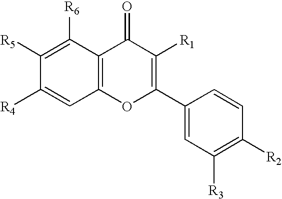

where R1, R2, R3, R4, R5, and R6 are one of the following groups: hydrogen, hydroxyl, and methoxyl. The flavonoids have a broad range of compounds. Some of the natural flavonoids are shown in Table 3.

Polyphenols

The polyphenols include resveratrol, kaempferol, and alkyl esters of gallic acids. They structures are given by the following formulas: Resveratrol (5-[(E)-2-(4-hydroxyphenyl)-ethenyl]benzene-1,3-diol)

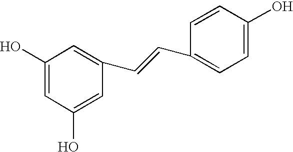

### Kaempferol (3,4′,5,7,-Tetrahydroxyflavone, or 3,5,7-Trihydroxy-2-(4-hydroxyphenyl)-4H-1-benzopyran-4-one)

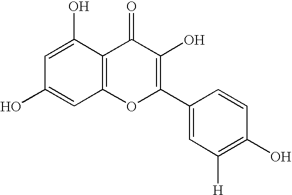

Alkyl ester of gallic acids:

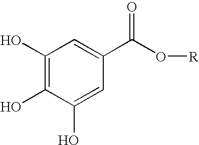

where R is an alkyl chain of 2-20 in length.

### Silibinin

Silibinin is the active constituent of silymarin extracted from milk thistle (Silybum marianum). Its structure is shown below.

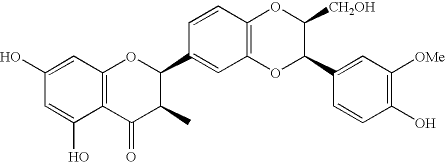

Complexes of silibinin, such as Silipide, a complex of silymarin and phosphatidylcholine (lecithin), silymarin-β-cyclodextrin, and glycosides of silibinin, which showed improved water solubility, are expected to have similar biological and therapeutic effects.

B. Pharmaceutical Carriers

The compounds can be administered orally, enterally, parenterally, topically, transdermally, subeutaneously, or using other standard routes of administration.

In one embodiment, the formations described above can be administrated orally in various dosage forms, such as pills (tablets, capsules, gels, gums, etc.) and syrup.

In another embodiment, the above formulations can be administrated by injection, infusion or nasal spray of a solution, for example, to control acute inflammation such as asthma and atherosclerosis.

In another embodiment, the formulations can be applied topically, in a dosage form of a solution, paste, gel, or patch, to a body area where protection or treatment is desired. For example, this product can be directly applied to the joints suffering from rheumatoid arthritis. This product can also be formulated in sunscreen lotion and applied to body to protect the skin from sunlight/UV irradiation.

In another embodiment, the formulations can be applied in a combination of dosage forms. For example, the product can be administrated orally for systemic effects and simultaneously applied topically for local effects.

To improve the efficacy of the treatment, the formulation can be manufactured in a delivery form to facilitate the delivery of the compounds to specific target sites, such as colon, brain and joints.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions. “Carrier” also includes all components of a coating composition which may include plasticizers, pigments, colorants, stabilizing agents, and glidants.

Formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6.sup.th Edition, Ansel et.al., (Media, Pa.: Williams and Wilkins, 1995) which provides information on carriers, materials, equipment and process for preparing tablets and capsules and delayed release dosage forms of tablets, capsules, and granules.

For injection, the formulation will usually consist of an effective dosage of the active ingredient in combination with a buffer such as buffered isotonic saline or sterile water.

For oral formulations, the active ingredient will typically be in the form of a capsule, tablet, beads, or liquid form. Examples of suitable coating materials for tablets include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit.RTM. (Roth Pharma, Westerstadt, Germany), Zein, shellac, and polysaccharides. The coating material may also contain conventional carriers such as plasticizers, pigments, colorants, glidants, stabilization agents, pore formers and surfactants.

Optical pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactase, sucrose, mannitol, sorbitol, cellulose, microcrystalline cellulose, kaolin, sodium chloride, dry starch, hydrolyzed starches pregelatinized starch, silicone dioxide, titanium oxide, magnesium aluminum silicate and powder sugar. Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol),polyethylene glycol, waxes, natural and synthetic hums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, methyl methacrylate copolymers, aminoalkyl methacrylate copolymers, polyacrylic acid/polymethacrylic acid and polyvinylpyrrolidone. Lubricants are used to facilitate tablet manufacture. Examples of suitable lubricants include, but are not limited to, magnesium stearate, calcium stearate, stearic acid, glycerol behenate, polyethylene glycol, take, and mineral oil. Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp). Stabilizers are sued to inhibit or retard drug decomposition reactions which include, by way of example, oxidative reactions.

Surfactants may be anionic, catioinic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether; polyoxyethylene tridecyl ether, polypropylene glycol butyl ether, Poloxamer.RTM, 401, stearoyl monoisopropanolamide, and polyoxyethylene hydrogenated tallow amide. Examples of amphoteric surfactants include sodium N-dodecyl-.beta.-alanin-c, sodium N-lauryl-.beta.-iminodipropionate, myristoamphoacetate, lauryl betaine and lauryl sulfobetaine. If desired, the tablets, beads granules or particles may also contain minor amount of nontoxic auxiliary substances such as wetting or emulsifying agents, dyes, pH buffering agents, and preservatives.

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate. Alternatively, extended release formulations can be prepared using osmotic systems or by applying a semi-permeable coating to the dosage form. In the latter case, the desired drug release profile can be achieved by combining low permeable and high permeable coating materials in suitable proportion. Devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Delayed release formulations are created by coating a solid dosage form with a film of a polymer which is insoluble in the acid environment of the stomach, and soluble in the neutral environment of small intestines. The delayed release dosage units can be prepared, for example, by coating a drug or a drug-containing composition with a selected coating material. The drug-containing containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrolintestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit.RTM. (Rohm Pharma; Westerstadt, Germany), including Eudragit.RTM. L30D-55 and L100 -55 (soluble at pH 5.5 and above), Eudragit.RTM. L-100 (soluble at pH 6.0 and above), Eudragit.RTM.S (soluble at pH 7.0 and above, as a result of a higher degree of esterification), and Eudragit.RTM. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies. The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthatlate, dibutyl phthalate, dibutyl sebacate, triethyl citrate, tributyl citrate, triethyl acetyl citrate, castor oil and acetylated monoglycerides.

A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective gliding is tale. Other glidants such as magnesium stearate and glycerol monasteries may also be used. Pigments such as titanium dioxide may also be used. Small quantities of an anti-foaming agent, such as a silicone (e.g., simethicone), may also be added to the coating composition.

Tablets are prepared by techniques commonly known in the art such as direct compression, wet granulation, or dry granulation processes. Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Formulations can also be prepared for administration topically or in transdermal patches, using methods known to those skilled in the art.

### II. Methods of Treatment

Methods for preventing, controlling and treating metabolic disorder including high blood cholesterol, triglyceride, and low density lipoprotein lipids (LDL), aging-related diseases including cardiovascular disease, rheumatoid arthritis, cancer, and Alzheimer's disease, and acute inflammatory disease including asthma, allergy, and sunburn have been developed.

A. Increasing Serum Bilirubin Level

Free radicals are one of the most important pathogens for inflammatory and age-related diseases. Thus, compounds having strong antioxidative activities and capable of scavenging free radicals are found to have therapeutic effects. Bilirubin is a potent endogenous antioxidant. However, the endogenous supply of bilirubin in the body diminishes gradually as human ages. The total serum bilirubin level of people over 50 years old can be as low as half of the level of people in ages 20-30. Smoking, environmental pollutants and physiological stress accelerate the diminishing of serum bilirubin. In one embodiment, the method of treatment involves direct replenishing of bilirubin. In another embodiment, the method of treatment is to administrate a compound or compounds which produce bilirubin through metabolic transformations. The compounds can be (i) a bilirubin pre-drug consisting of heme, hematin, hemin, and/or its protoporphyrin analogues; (ii) derivatives and/or analogues of bilirubin; (iii) tripyrroles; and/or (iv) dipyyroles. In another embodiment, the treatment is to reduce the excretion of bilirubin with a compound or compounds that inhibit bilirubin UDP-glucuronosyltranferase (UDPGT) enzymes.

Specifically, the method increases or maintains the level of total serum bilirubin to an optimal health and/or therapeutic ranges, which for men is generally 0.5-4 mg/dL (or 8-65 μmol/L), preferably 0.8-3.0 mg/dL (or 13.5-50 μmol/L), and most preferably 1.0-2.5 mg/dL (or 15-40 μmol/L); and for women generally 0.3-3.0 mg/dL or 5-50 μmol/L, more preferably 0.5-2.5 mg/dL or 12-40 μmol/L, and most preferably 0.8-2.0 mg/dL or 12-35 μmol/L.

B. Inhibition of Enzymes Involved in Fat/Cholesterol Absorption and Biosynthesis

Metabolic disorders (i.e., high blood cholesterol and triglyceride concentrations) can be controlled by either reducing de novo biosynthesis of cholesterol and triglyceride in the liver or limiting the absorption of fat and cholesterol from food/diet sources.

The synthesis of fat and cholesterol is controlled by 3-1-hydroxylacyl-CoA dehydrogenase, enoyl-CoA reductase and hydroxymethylglutaryl-CoA reductase (“HMG-CoA reductase”). These fat and cholesterol synthesis enzymes require NADH and NADPH, cofactors for their enzyme activities. Malic dehydrogenase, isocitrate dehydrogenase, and glucose-6-phosphate dehydrogenase are considered the major NADPH₂ and NADH producing enzymes. Therefore, the method of treatment is to reduce the activity or amount or both the activity and amount of the enzymes that control the fat and cholesterol synthesis and the enzymes that supply the co-factors.

The absorption of fat and cholesterol from intestine is controlled by pancreatic enzymes including lipase, phospholipase and cholesterol esterase. Thus, in another embodiment, the method of treatment is to reduce the absorption of fat and cholesterol from the gastrointestinal system by inhibiting pancreatic enzymes.

It was discovered that bilirubin and its derivatives can modulate the activity of the enzymes involved in both the absorption and the de novo synthesis of fat and cholesterol. Therefore, the treatment is expected to be highly effective to lower the levels of total blood cholesterol, triglyceride and LDL-cholesterol.

C. Inhibition of Enzymes Involved Inflammatory Reactions

One of the most important pathogenic factors in age-related diseases such as atherosclerosis, stroke and cancer, and autoimmune diseases such allergy, rheumatoid arthritis and organ rejection in transplant, is inflammation. In general, inflammatory reactions cascade through the following successive process:

(i) generation and/or buildup of free radicals. Reactive oxidative species (ROS), including superoxide anion, hydroxyl radicals, and hydrogen peroxide, can be generated in the endogenous metabolism processes or by activated immune and inflammatory cells. Other free radical species may come from the environment such as cigarette smoking or air pollution. Free radicals are hyperactive and can direct damage tissues or organs, causing problems such as stroke and atherosclerosis.

(ii) induction of oxidative stress, expression of stress proteins and their release from tissues and migrating cells. One of the enzymes involved in the initial inflammatory reaction is phospholipases A2 (PLA2). PLA2 cleaves arachidonic acids from the backbone of phospholipids, releasing arachidonic acid which is the precursor of inflammatory mediators of leukotrienes and prostaglandins. Additionally, PLA2 is also related to platelet-activating factors (PAF) which induces inflammatory reactions in various animal species and in human tissue. PAF mimics the main clinical features of asthma and is particularly effective in producing hyperreactivity and accumulation of eosinophils in lung tissue.

(c) cascading inflammatory reactions. The inflammatory enzymes such as phospholipase A2, lipooxygenase and cyclooxygenase then catalyze the inflammation reactions. For example, oxidation of arachidonic acid by lipoxygenases results in the formation of leukotrienes, a class of compounds that are potent bronchoconstrictors. They are involved in asthma, anaphylactic shock, and rheumatoid arthritis. Oxidation of arachidonic acid by cyclooxygenase (COX) produces prostacyclins, thromboxanes, and prostaglandins. Prostacyclins are powerful vasodilators and inhibitors of platelet aggregation. Thromboxanes (TXA2) cause platelet aggregation and vasoconstriction. These prostaglandins exert a wide range of effects on different parts of the body. In short, these mediators initiate, amplify and perpetuate the inflammation disease state by the oxidation of nucleic acids, proteins, and membrane lipids.

Corticosteroids prevent the formation of PGs by causing the release of lipocortin, which inhibits phospholipase A2 to reduce arachidonic acid release. NSAIDs work by inhibiting cyclooxygense activity and expression. These drugs may cause serious complications like gastric ulcers, depressive disorders and suicidal tadencies, and respiratory complications.

Useful compounds are effective to scavenge the free radicals and inhibit both phospholipase A2 and cyclooxygenase. The combined cellular mechanisms of the compounds should have potent anti-inflammatory effects.

D. Inhibition of Enzymes Involved in Aging Process

Alzheimer's disease (AD) is a complex age-related neurodegenerative disease. The deficit in cholinergic transmission, which is associated with acetylcholinesterase has been shown to play a primary role in the pathogenesis of AD. Inhibition of of acetylcholinesterase (AChE) activity, thus prolonging the action of acetylcholine at the postsynaptic cholinergic receptor and enhancing cholinergic function, has beneficial effect on AD.

Regulation of gene expression is mediated by several mechanisms such as DNA methylation, ATP-dependent chromatin remodeling, and post-translational modifications of histones, which include the dynamic acetylation and deacetylation of epsilon-amino groups of lysine residues present in the tail of core histones. The enzymes responsible for reversible acetylation/-deacetylation processes are histone acetyltransferases (HATs) and histone deacetylases (HDACs), respectively. Histone acetylation and deacetylation play important roles in the modulation of chromatin topology and the regulation of gene transcription. Recent studies have shown that histone deacetylase inhibition is a new way to control the proliferation of tumor cells by inducing cell cycle arrest, differentiation, and apoptosis of tumor cells.

Useful compounds are strong inhibitors for acetylcholinesterse and histone deacetylase. It is expected that medical formulations consisting of the compounds will have therapeutic effects on Alzheimer's disease and cancer as well.

E. Inhibition of Bilirubin-UDPGT Enzymes

The human UDP-glucuronosyltransferase (UDPGT) isozymes in the GI tract participate in the broad and critical function of detoxifying lipid-soluble toxins derived metabolically or ingested as part of the diet and/or as medications. By conjugation of glucuronic acid to the acceptor substrate, they convert the lipophile to inactive glucuronides that are then readily excreted in urine or feces. However, the same function of the UDPGTIAI enzymes apparently have a disadvantageous effect on the therapeutic formulations described above, since they can be cleared from the body prematurely by the UDPGT enzymes.

Therefore, in one embodiment, the method of treatment is to reduce bilirubin secretion by administrating a compound or compounds that inhibit bilirubin UDP-glucuronosyltranferase (UDPGT) enzymes. It has been discovered that structural analogues of bilirubin are strong competitive inhibitors of UDPGT enzymes.

### EXAMPLES

The present invention will be further understood by reference to the following non-limiting illustrative examples.

### Example 1

### Inhibition of Hydrolases and Esterases by Bilirubin

The inhibitory effects of bilirubin on hydrolases and esterases are illustrated in this example.

Materials and Methods

Pancreas phospholipase A2 (PLA2) (SigmaAldrich Cat.# P223), acetylcholinesterase (SigmaAldrich cat.# C0663), acetyleholine chloride (Cat.# A6625), and bilirubin (SigmaAldrich #B4126) were purchased from SigmaAldrich. Bilirubin solution was prepared freshly by dissolving it in dimethyl sulfoxide (DMSO) and further dilutions were made in Tris-HCl buffer (pH 7.4). The sPLA2 activity was determined by the FlashPlate assay procedure described by Do and Kasila (American Biotechnology Laboratory, June 2001, P 51-52). Briefly, phospholipid flash plates (PerkinElmer Life Sciences, cat. #SMP108) were coated with 0.2 mL/well of the substrate of 1-steroyl 2-arachidonyl phosphatidylcholine (PerkinElmer Life Sciences, cat. #NE872). The plate was covered and incubated overnight at room temperature. After the incubation, the wells were aspirated prior to use in the PLA2 assay. The substrate assay buffer contained 50 mM tris-HCl (pH=7.3), 0.2 M NaCl, 0.1 mM CaCl₂, 0.1 mM EDTA, 0.02% deoxycholate, 10-50 units/mL PLA2, and final volume 200 μL/well. The bound radioactivity was determined by counting the FlashPlate.

Since bilirubin itself gives an absorption peak at 460 nm, which interferes with the cholinesterase assay procedures in many standard assay kits, a modified method was developed. The acetylcholinesterase activity was determined by measuring the quantity of choline release from acetylcholine:

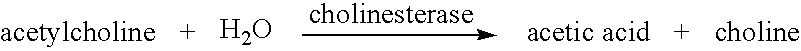

Briefly, after 30 minutes reaction in the above acetylcholine-enzyme solution at pH=7.8, in the absence or presence of bilirubin, the reaction was stopped by heating to about 100° C. in boiling water. The solution was then cooled down to 37° C. Residual bilirubin was neutralized by titrating with H₂O₂. The amount of choline was then determined by the following choline oxidase assay method provided by SigmaAldrich (Cat.# C5896):

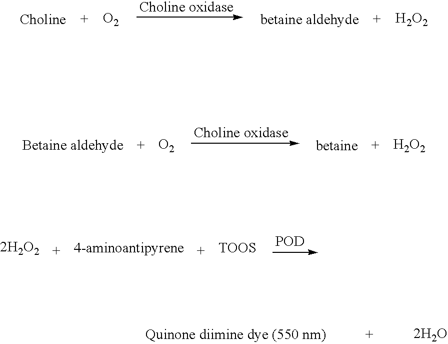

Where TOOS is n-ethyl-n-(2-hydroxyl-3-sulfopropyl)-m-toluidine.

Results

As shown in FIG. 1, bilirubin inhibits the panereatic phospholipase A2 (PLA2) and cholesterol esterase (CHE) in a dose dependent manner. At a bilirubin concentration of about 10 mg/L, which would be considered as moderately high if it was measured for total serum bilirubin, an inhibition of about 80% of the PLA2 activity and about 50% of the CHE activity was obtained. In a similar manner, bilirubin also inhibits the cholesterol esterification by liver cholesterol esterase. PLA2 plays important roles in at least two health aspects. First, PLA2 in tissues/membranes is responsible for the release of arachidonic acids from hydrolysis of phospholipids in membranes, the first step of the cascade of the inflammatory reactions, as described above. Thus inhibition of PLA2 activity will be of therapeutic significance in preventing and controlling inflammation. The inhibitory effect of bilirubin demonstrates that bilirubin has potent anti-inflammatory effect. Second, pancreatic PLA2 in the gastrointestinal tract is responsible for the emulsification of fat and cholesterol ingested from food, facilitating the absorption of fat and cholesterol from intestine. Inhibition of PLA2 would thus reduce the absorption of fat and cholesterol from the gastrointestinal system, which would avoid the overload of fatty food to the human body. Similarly, the inhibition of cholesterol esterase by bilirubin would also reduce the absorption of cholesterol esters from the intestine system.

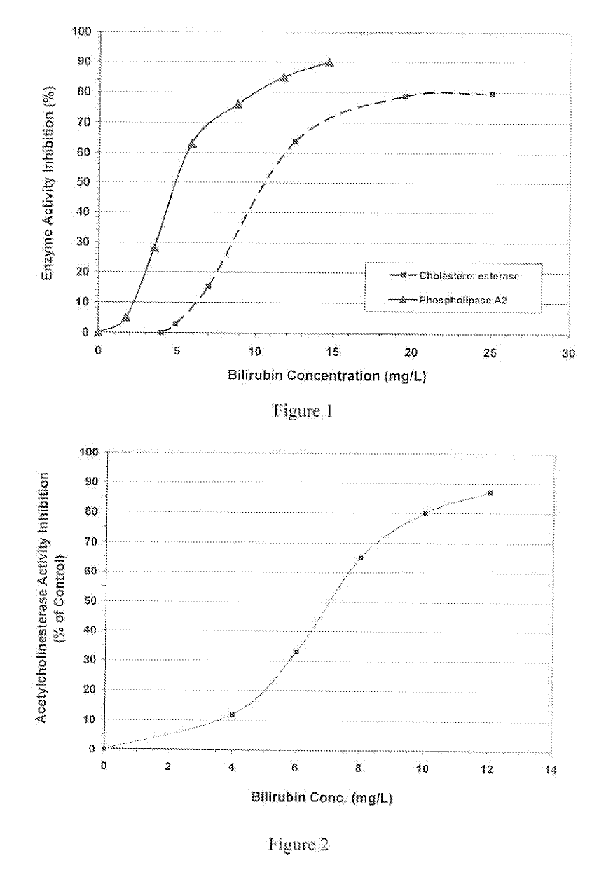

FIG. 2 shows the inhibition of acetylcholinesterase activity by bilirubin. High acetylcholinesterase activity has been found in the brain of Alzheimer's disease patients and inhibition of the brain acetylcholinesterase has been shown to be at least partially effective in preventing and treating the disease. Bilirubin shows a dose-dependent inhibitory effect on acetylcholinesterase. At high bilirubin concentrations, the acetycholinesterase activity can be completely eliminated. Therefore, bilirubin should be useful in prevention and treatment of Alzheimer's disease.

### Example 2

### Inhibition of Cyclooxygenase (COX)

Materials and Methods

The activity of COX was determined by the procedure described in Assay Design's Enzyme Immunometric Assay (EIA) kit (Assay Design Inc., TiterZyme EIA cat.#900-094). Briefly, the kit uses a monoclonal antibody to human COX-II immobilized on a microtiter plate to bind the human COX in the standard or sample. After a short incubation the excess standard or sample is washed out and a rabbit polyclonal antibody to human COX labeled with the enzyme. Horseradish peroxidase is added. This labeled antibody binds to the human COX captured on the plate. After a short incubation the excess labeled antibody is washed out and substrate is added. The substrate reacts with the labeled antibody bound to the human COX captured on the plate. After a short incubation, the enzyme reaction is stopped and the color generated is read at 450 nm. The measured optical density is directly proportional to the concentration of human COX.

Purified cyclooxygenases (COX) were obtained from Kayman Chemical Company and used to examine the inhibitory effect of bilirubin with arachidonic acid as the substrate. High purity of bilirubin (SigmaAldrich #B4126), biliverdin (Frontier Scientific Inc., Cat. B655-9) and hemin (SigmaAldrich, #51280) were used as received.

Results

FIG. 3 shows the COX inhibition as a function of bilirubin concentration. Bilirubin inhibits the COX activity in a concentration-dependent manner. Up to 70% inhibition of the COX activity is obtained at a concentration of 10 mg/L bilirubin. At total serum bilirubin level comparable to this concentration, the risk order of cardiovascular disease (CVD) is considerably reduced.

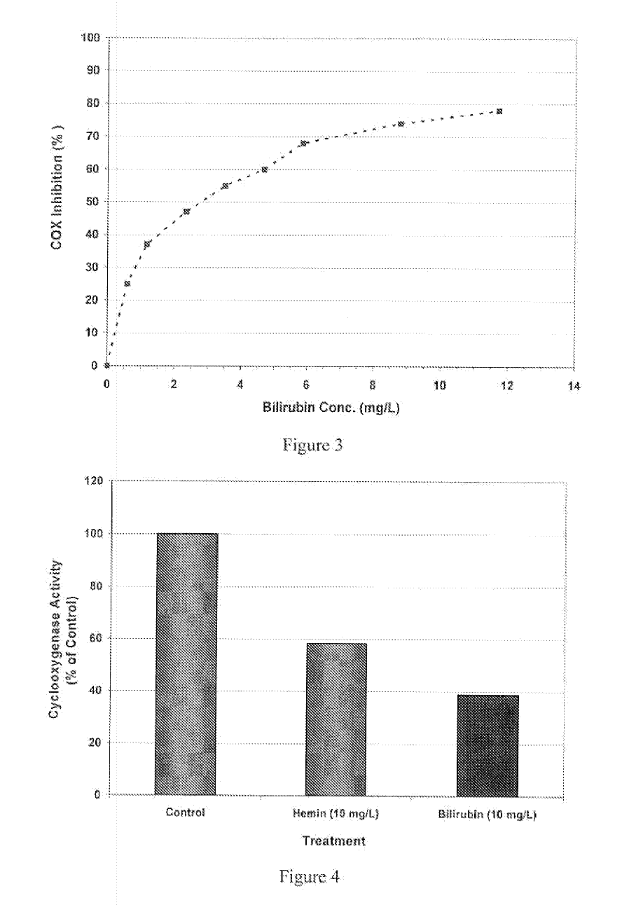

FIG. 4 presents a comparison of the inhibitory effects of bilirubin and hemin. At a concentration of 10 mg/L, both bilirubin and hemin are effective in reducing COX activity. This example illustrates that heme, the bilirubin pre-drug, has similar effects to bilirubin in inhibiting cyclooxygenase. It is expected that the derivatives and analogues of bilirubin would have similar or even more potent therapeutic effects when compared to bilirubin.

### Example 3

### Inhibition of Fat and Cholesterol Synthesis Enzymes

Materials and Methods

NAD (Cat.#N1511), NADH (Cat. N9410), NADP (Cat.#N8610) and NADPG (Cat. #N7505), HMG-CoA (Cat.#H6132) were purchased from Sigma Aldrich (St. Louis, Mo.). NADP-linked isocitrate dehydrogenase was also obtained from SigmaAldrich (Cat.#I2516). Other liver enzymes were prepared as follows.

Liver microsomes, which are used to investigate cholesterol metabolism, were prepared as follows. Liver was homogenized in 50 mM Tris HCl buffer (pH 7.4), containing 0.3 M sucrose, 10 mM DTT, and 10 mM EDTA. The homogenate was centrifuged at 20,000×g for 15 min, and the supernatant was centrifuged at 100,000×g for 60 min. The resulting microsomal fraction was suspended in 3 ml of 0.1 M potassium phosphate buffer (pH 7.4), containing 1 mM EDTA and 5 mM DTT. Aliquots were immediately frozen in liquid nitrogen and stored at −20° C. until analysis.

Assay of HMG-CoA reductase. Microsomal suspensions of 500 ml, containing 5 mg protein, were preincubated for 5 min at 37° C. with 450 ml of 0.1 M potassium phosphate buffer (pH 7.4), containing 1 mM EDTA and 12 mM glucose-6-phosphate. The assay was initiated by adding 50 ml of cofactor-substrate solution (0.1 mM HMG-CoA, 3 mM MADPH, and 2 U/ml glucose-6-phosphate dehydrogenase). The incubation was performed at 37+ C. for 30 min and was terminated by the addition of 50 ml of 1 M MaOH. After lactonization, the mevalonolactone was separated by thin layer chromatography. The eluate, which contained mevalonolactone, was collected in a scintillation vial. The radioactivity was then counted in a Packard 3320 scintillation spectrometer.

Results

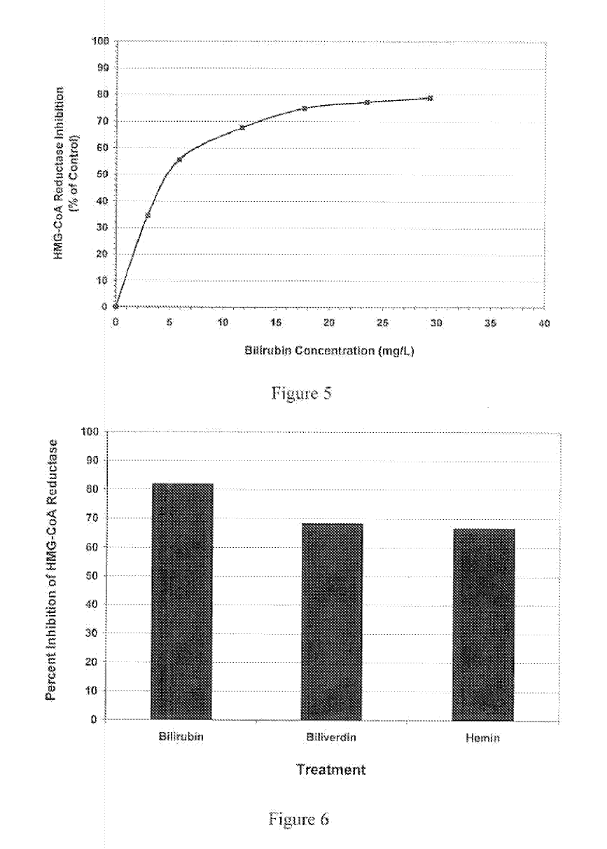

The enzyme HMG-CoA reductase (mevalonate:NADP oxidoreductase, ED1.1.1.34) is considered to be the rate-limiting enzyme of cholesterol biosynthesis in liver and intestinal mucosa. As shown in FIG. 5, bilirubin is a potent inhibitor of HMG-CoA reductase. At 10 mg/L, bilirubin reduces the HMG-CoA reductase activity by as much as 60%. The inhibition effect increases with increasing bilirubin concentration. Hematin, the pre-drug of bilirubin, and biliverdin both strongly inhibit the activity of HMG-CoA reductase under moderately high concentrations, as compared in FIG. 6.

The redox state of the blood solution, as measured by the NAD/NADH ratio, also has a very significant influence on the de novo synthesis of fat and cholesterol. The synthesis of fats and cholesterols is favored under reducing conditions (i.e., small ratios of NAD/NADH and NADPH/NADPH₂). Since glucose-6-phosphate dehydrogenase (G6PDH), 6-phosphogluconate dehydrogenase (PGDH), malic dehydrogenase (MDH), and isocitrate dehydrogenase (iCDH) are considered to be the major enzymes producing NADH and NADPH₂, compounds that inhibit these enzymes will reduce the generation of NADH and NADPH₂ in the body, and should reduce the de novo synthesis of fats and cholesterols.

Bilirubin is a potent inhibitor for these enzymes. As shown in FIG. 7, isocitrate dehydrogenase and malate dehydrogenase are inhibited by bilirubin in a dose-dependent manner. At a concentration equivalent to the moderate level of serum bilirubin, the enzymes are strongly suppressed. Similar inhibitory effects have been observed on G6PDH.

### Example 4

### Inhibition of Bilirubin Glucuronidation

Materials and Methods

UDP-glucuronosyltransferase 1A1 isozenzyme (Cat. #U7254), uridine-5′-diphosphoglucuronic acid (UDPGA, #U5625), bilirubin (B), naringenin (N5893), octyl gallate (#48700) and quercetin (Q0125) were purchased from SigmaAldrich (St. Louis, Mo.). Bilirubin dimethyl ester (#B612-9), biliverdin dimethyl ester (#B610-9), biliverdin hydrocholoride (#B655-9), mesobilirubin (#M588-9), urobilin hydrochloride (#U590-1), and stercobilin hydrochloride (#S594-9) were purchased from Frontier Scientific Inc. (Logan, Utah).

The enzyme activity assay method was based on those of Adlard and Lathe (Biochem. J., 1970, 119:437-445). Incubation mixtures (total vol. 1.25 ml, pH 7.9) were prepared in tubes kept in ice and had the following composition: bilirubin (0.1 mM), UDP-glucuronic acid (0.25 mM), serum (5.8%, v/v), Mg2+ (5.0 mM), microsomal preparation (0.5-1.5 mg of protein), 10 mM tris-0.14 M KCl (pH 8.0), and 10% (v/v) dimethylsulphoxide. Incubation (10 min, 37° C.) was carried out with shaking (100 cycles/min). After incubation, tubes were placed in ice-water (5 min) and direct-reacting bilirubin was determined. All incubations were done in duplicate. Enzyme activity was expressed as μg of bilirubin conjugated/h per mg of protein. Differences from controls of more than 10% were regarded as significant.

Results

The inhibitory effects of the selected compounds on the bilirubin-UDPGT enzyme (UDPGT1A1) are shown in Table 4. The derivatives and analogues of bilirubin are strong competitive inhibitors of bilirub-UDPGT enzymes. For example, dimethoxybilirubin has an inhibition constant (Ki) of about 1 μm. several other classes of substances, including flavonoids and phenols, also inhibit bilirubin glucuronidation by UDPGT1A1 in a dose-dependent manner. For example, naringenin has an IC50 of about 16 μmol/L and apparent Ki of 5 μm under the testing conditions. The compounds are mostly reversible competitive inhibitors, since it was found that an increase of bilirubin concentration reduces the inhibition degree. These substances should reduce the excretion of bilirubin, thus facilitating the body attaining an optimal level of bilirubin. They should also reduce the elimination of bilirubin from the intestine prematurely when it is administrated orally.

### Example 5

### Inhibition of Acute Inflammation

The acute reactions of human skin to solar ultraviolet (UV) radiation (290-400 nm) are recognized as a form of inflammatory reactions. Studies have shown that reactive oxygen species (ROS) and inflammatory mediators (prostaglandins and leukotrienes) are produced when tissues are exposed to UV. The in vivo effectiveness of bilirubin inhibition on acute skin inflammation induced by UV irradiation was demonstrated in this example.

Materials and Methods

Healthy volunteers were exposed to solar radiation for evaluation of minimal erythema dose (MED)—a direct sign of acute inflammation, without and with pretreatment of bilirubin (dissolved in dimethyl sulfoxide).

Results

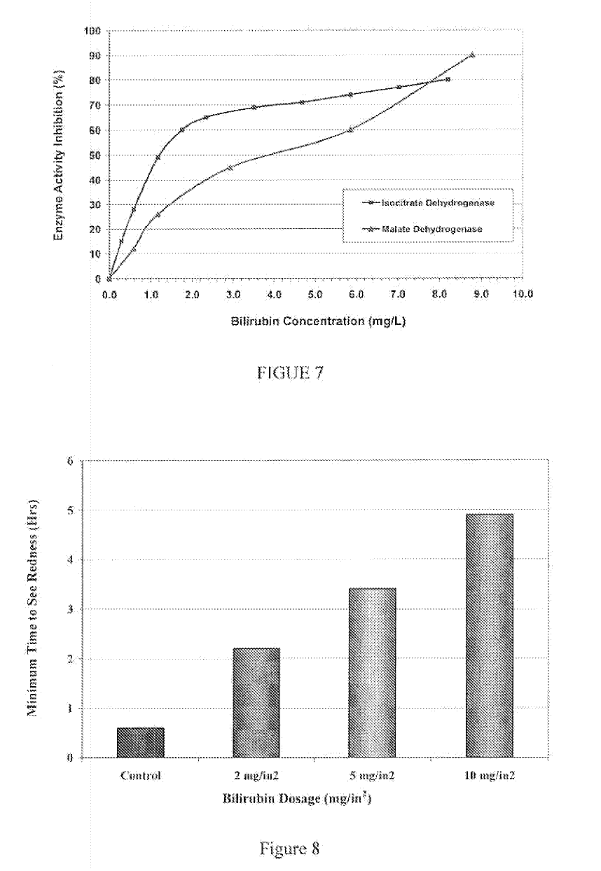

As shown in FIG. 8, the MED was increased by bilirubin treatment in a dose-dependent manner. Untreated skins developed severe sunburn-like rash after a short time of UV exposure, while bilirubin treated skins area showed no sign of inflammation.

### Example 6

### Inhibition of Histone Deacetylase and Cell Growth

The inhibitory effects of bilirubin on histone deacetylase activity and tumor cell growth are demonstrated in this example.

### Materials and Methods

Cell growth. Human carcinoma cells were cultured continuously in a medium containing ferrous sulfate, kept in a humidified incubator at 37° C. Cancer cells were seeded in triplicate at a density of 10,000 cells/3.5-cm culture dish overnight before being subjected to treatments. Growth was estimated by counting cells in a hemocytometer after staining cells with trypan blue. Cell yields from cultures maintained in seeding medium served as growth controls for computing the relative cell growth.

Assay of histone decaetylase (HDAC). The activity of HDAC was measured according to the procedure provided by SigmaAldrich (Histone Deacetylase Assay Kit, Cat.# CS1010). HeLa cel lysate provided by the assay kit was used as a source for HDAC activity in the inhibition testing.

Results

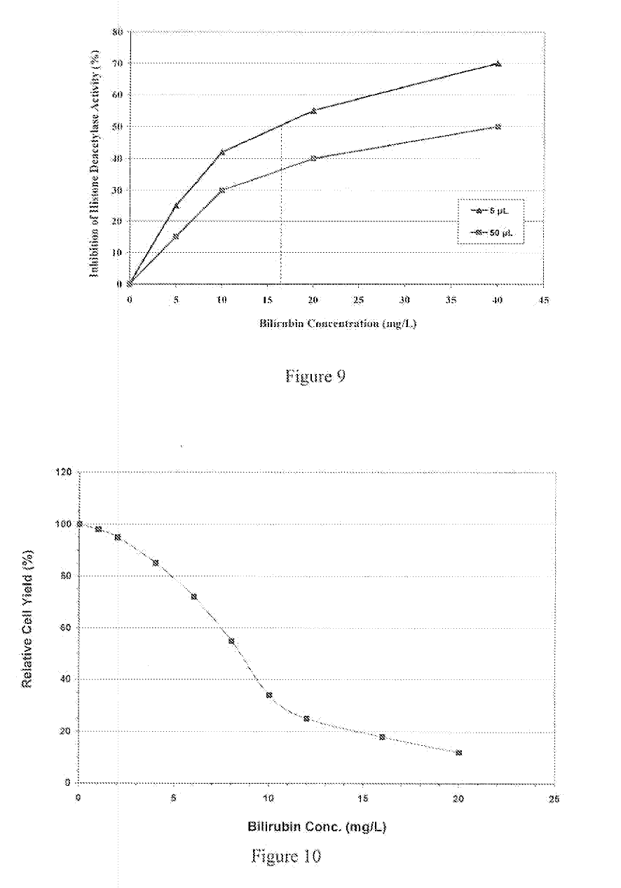

As shown in FIG. 9, bilirubin inhibits the activity of histone deacetylase in a dose-dependent manner. The inhibition also depends on the substrate concentration. The 50% inhibition concentration (IC50) increases from 16.5 mg/L to 40 mg/L when the substrate concentration is increased from 5 μM to 50 μM.

FIG. 10 shows relative cell growth versus bilirubin dosage for the tumor cell growth. It can be seen that at concentrations above 10 mg/L, the cell growth was largely suppressed. The results demonstrate that bilirubin inhibits the growth of cancer cells.

### Example 7

### Using Bilirubin to Control Blood Cholesterol

This example demonstrates the in vivo therapeutic effectiveness of the methods and formulations described above.

Methods and Materials

2-6 bilirubin pills (10 mg/capsule)were ingested daily as a dietary supplement over time period from 3-12 months. Blood samples were taken by medical professionals and blood chemistry, including serum bilirubin level, total cholesterol, triglyceride, LDL-cholesterol, and HDL cholesterol were analyzed by independent medical testing laboratories.

Result

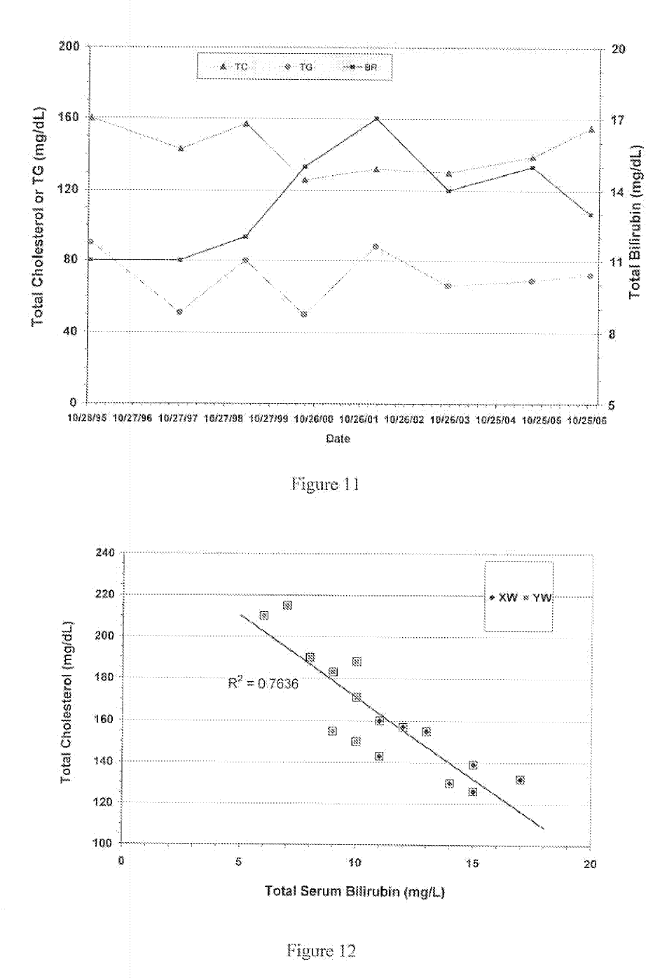

FIG. 11 shows the levels of total serum bilirubin, total cholesterol and triglcyerides over the testing period for the author. The results demonstrate that administration of bilirubin effectively increased the total serum bilirubin level. Correspondingly, the total cholesterol concentration varied inversely. As shown in FIG. 12, a higher serum bilirubin level leads to lower levels of total cholesterol, LDL-cholesterol and triglycerol. Tests also showed that the fecal fat content was substantially increased by bilirubin treatment, demonstrating that one mechanism of lowering the total blood cholesterol by bilirubin is reduced absorption of fat from the food.

### Example 8

### New Risk Factor of Cardiovascular Disease

High blood cholesterol level and the ratio of total cholesterol over high density lipoprotein (HDL) cholesterol, TC/HDL, are two established risk factors for cardiovascular disease. However, studies have also shown that these risk factors sometimes provide poor prognosis for CVD. A large percentage of atherosclerosis and stroke patients had a total cholesterol level below the defined threshold for cardiovascular disease.

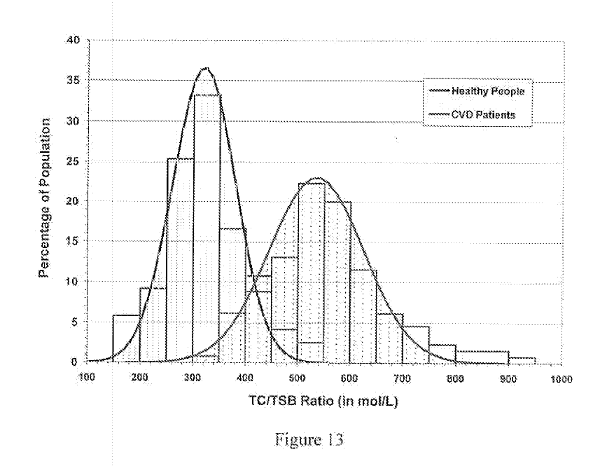

The ratio of total cholesterol (TC) over total serum bilirubin (TSB) concentration, TC/TSB, has been determined to be an independent and reliable risk factor for CVD, FIG. 13 shows the distribution of the TC/TSB ratio for healthy people (more 17,000 people) and CVD patients (>15,000 people) from various parts of China. The statistical analysis results demonstrated that cardiovascular disease patients have a much higher TC/TSB ratio than the healthy people. The average TC/TSB ratio of the CVD patients averaged at 535 (where TC and TSB were expressed in mmol/L), while that of the healthy people had an averaged of 320. Furthermore, the two distribution curves for the two groups have only a small overlap, indicating that the new risk factor would provide a reliable prognosis for CVD. The results show that when the TC/TSB ratio is above 400, there is a high risk of CVD. Furthermore, the higher the ratio, the greater the risk of have CVD problems.

Modifications and variations will be obvious to those skilled in the art from the foregoing detailed description. Such modifications and variations are intended to come within the scope of the appended claims.

TABLE 1
Examples of bilirubins and their structural analogues

| | |
|---|---|
| General structure: | 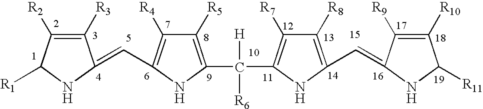 |

| Name | Structure Formula |
|---|---|
| Bilirubin (IXα) | 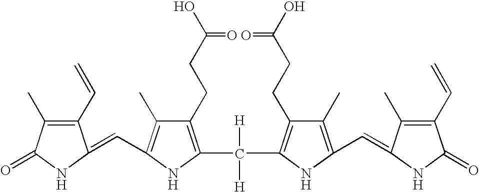 |
| Bilirubin (XIIIα) | 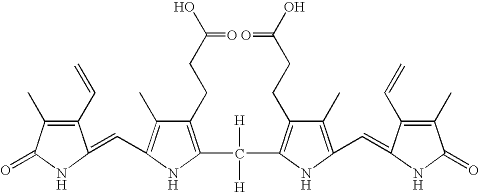 |
| Bilirubin (IIIα) | 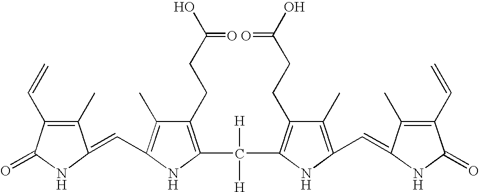 |
| Bilirubin (IVα) | 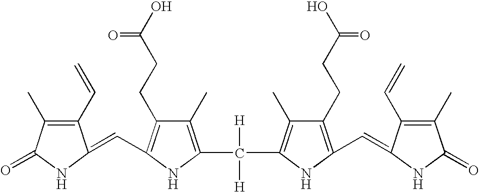 |
| Mesobilirubin (IVα) | 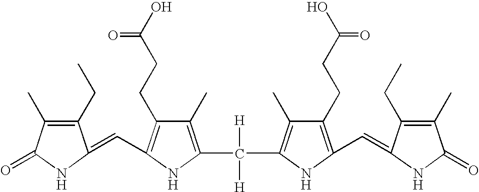 |
| Bilirubin (IXβ) | 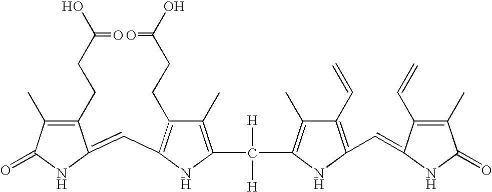 |
| Bilirubin (VIIγ) | 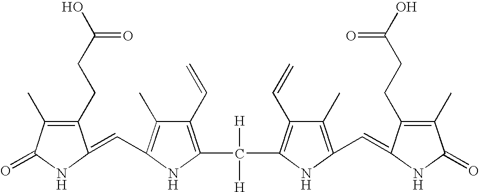 |
| Bilirubin (VIIIγ) | 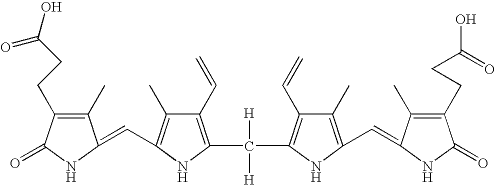 |
| Bilirubin with aliphaticfatty acids. where R hasa carbon length of 2-20. | 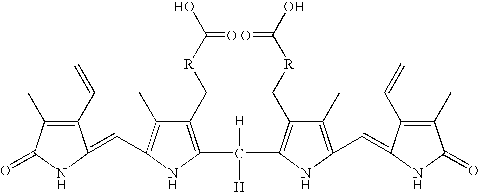 |
| Dimethylbilirubin (IXα) | 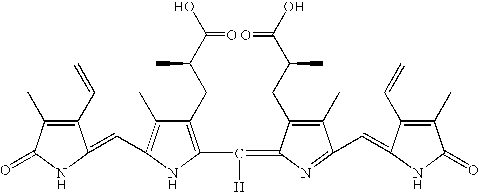 |
| Tetramethylbilirubin (IXα) | 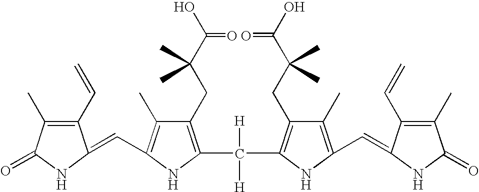 |
| Dimethoxylbilirubin | 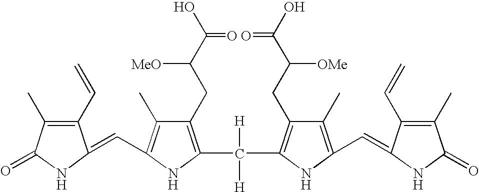 |
| Carboxylphenylbilirubin | 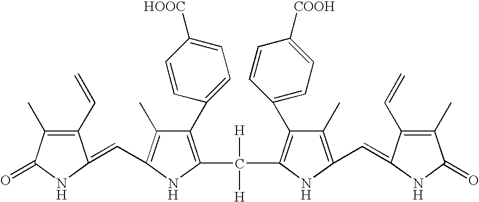 |
| 3,18-diphenyl bilirubin(VIIIα) | 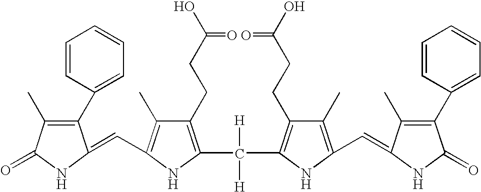 |
| Bilirubin 8,12-di-ethylsulfonic acid | 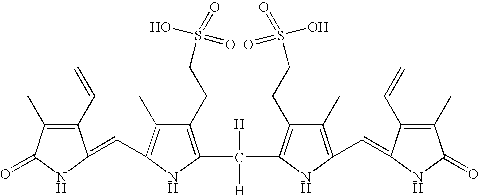 |
| Bilirubin 3,18-di-dimethylsulfate | 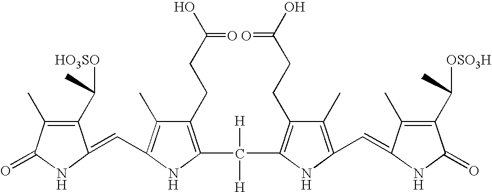 |
| Bilirubin 8,12-di-hexylhydroxamic acid | 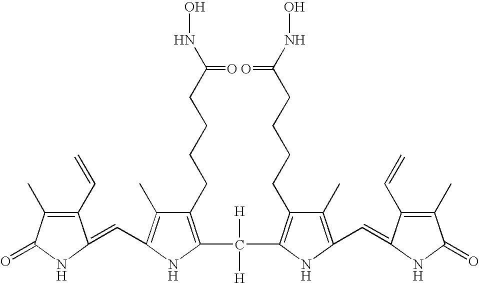 |
| Dimethoxy bilirubin | 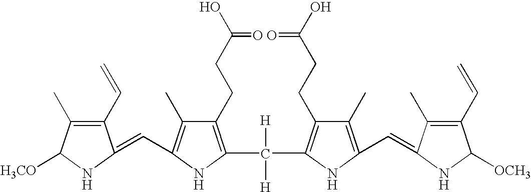 |
| Difluorobilirubin(XIIIα) | 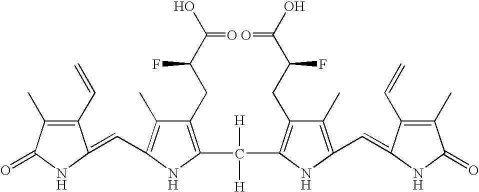 |
| 10-Methyl-bilirubin | 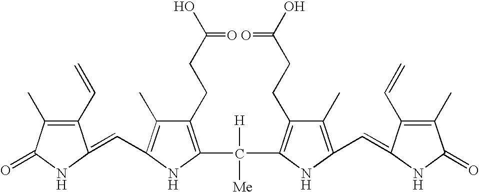 |
| 10-phenyl-bilirubin(XIII). | 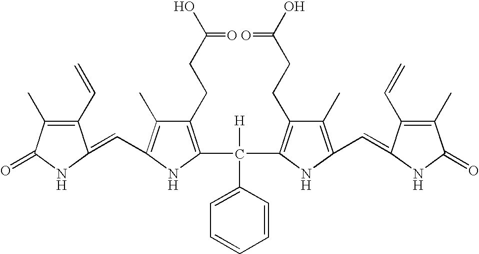 |

TABLE 2
Examples of biliverdins and their structural analogues

| | |
|---|---|
| General formula: | 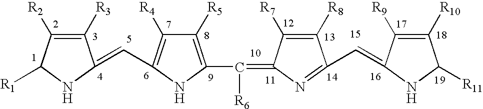 |

| Name | Structure Formula |
|---|---|
| Biliverdin (IXα) | 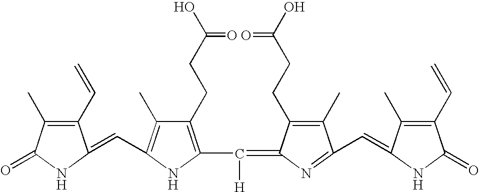 |
| Biliverdin (XIIIα) | 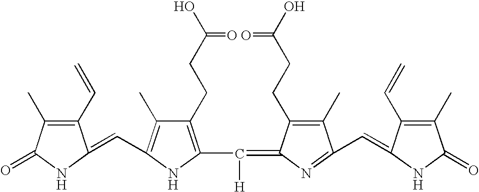 |
| Biliverdin (IIIα) | 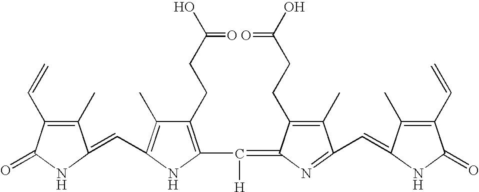 |
| Biliverdin (IVα) | 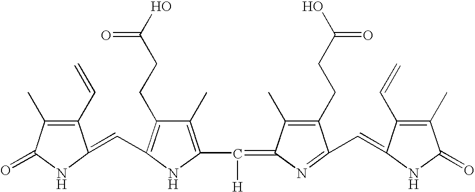 |
| Mesobiliverdin (IVα) | 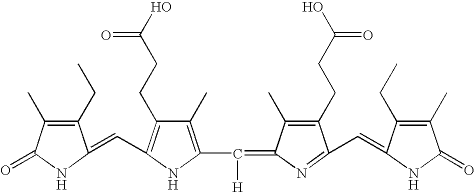 |
| Biliverdin (IXβ) | 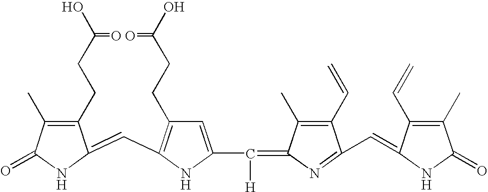 |
| Biliverdin (VIIγ) | 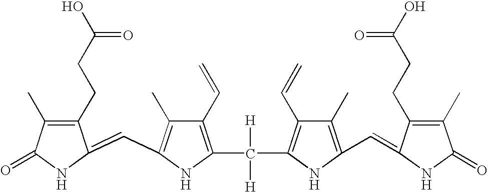 |
| Biliverdin (VIIIγ) | 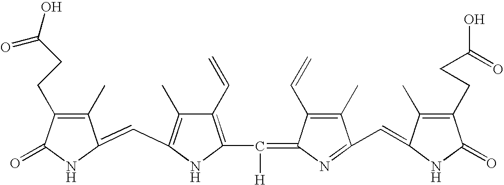 |
| Biliverdin with aliphaticfatty acids, where R has acarbon length of 2-20 | 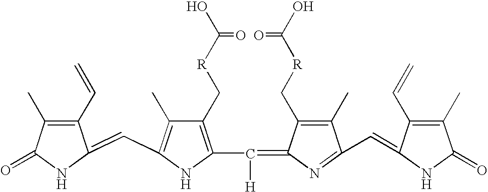 |
| Dimethylbiliverdin (IXα) | 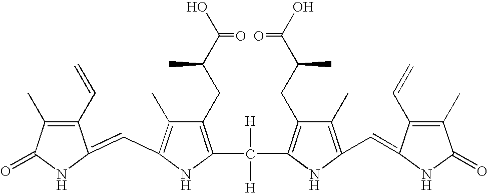 |
| Tetramethylbiliverdin(IXα) | 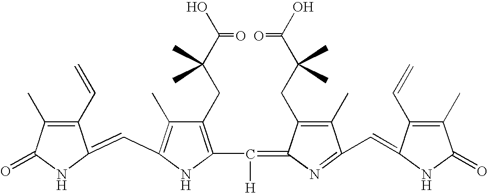 |
| Dimethoxyl biliverdin | 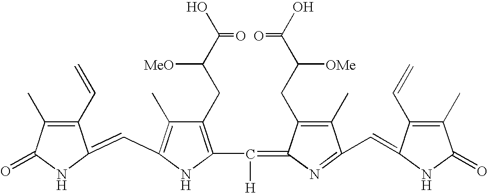 |
| Carboxylphenylbiliverdin | 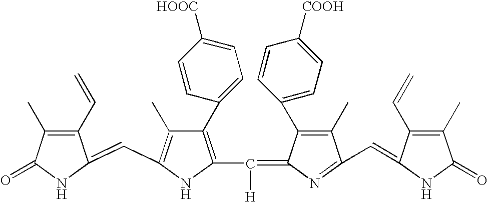 |
| 3,18-diphenyl biliverdin(VIIIα) | 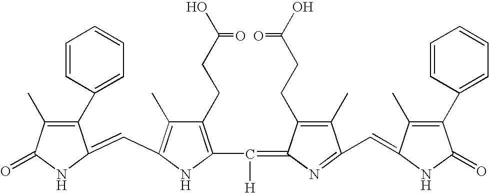 |
| Bactobilin (urobiliverdin)(where X is carboxlicacid, —CO₂H) | 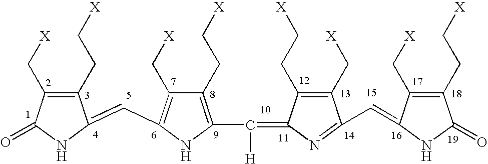 |
| Coprobiliverdin(where X is carboxlicacid, —CO₂H) | 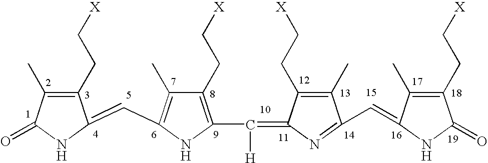 |

TABLE 3
Examples of natural flavonoids and polyphenols

| Name | Systematic Name | Structure Formula |
|---|---|---|
| Naringenin | (±)-2,3-Dihydro-5,7-dihydroxy-2-(4-hydroxyphenyl)-4H-1-benzopyran-4-one; 4′,5,7-Trihydroxyflavanone | 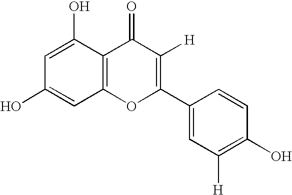 |
| Chrysin | 5,7-Dihydroxyflavone | 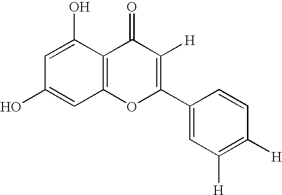 |
| Apigenin | 4′,5,7-Trihydroxyflavone;5,7-dihydroxy-2-(4-hydroxyphenyl)-4H-1-benzopyran-4-one | 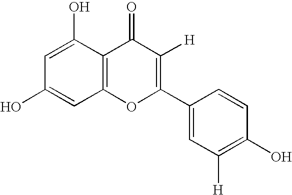 |
| Hesperetin | 3′,5,7-Trihydroxy-4-methoxyflavanone;(S)-2,3-dihydro-5,7-dihydroxy-2-(3-hydroxy-4-methoxyphenyl)-4H-1-benzopyran-4-one | 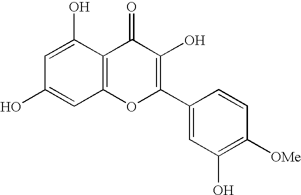 |
| Acacetin | 5,7-Dihydroxy-4′-methoxyflavone | 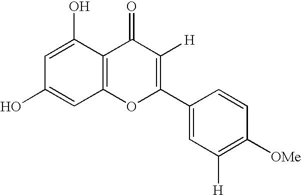 |
| Tangeretin | 5,6,7,8-tetramethoxy-2-(4-methoxyphenyl)-4H-1-benzopyran-4-one | 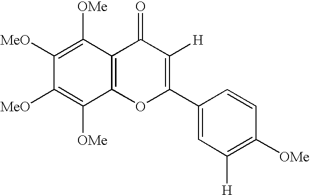 |
| Quercetin | 2-(3,4-Dihydroxyphenyl)-3,5,7-trihydroxy-4H-1-benzopyran-4-one | 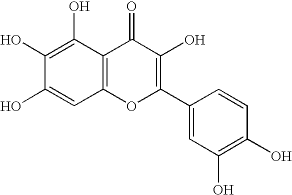 |
| Galangin | 3,5,7-Trihydroxyflavone | 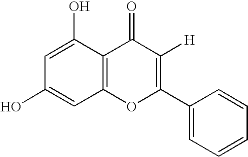 |
| Kaempferolrobigenin) | 3,4′,5,7-Tetrahydroxyflavone;3,5,7-Trihydroxy-2-(4-hydroxyphenyl)-4H-1-benzopyran-4-one | 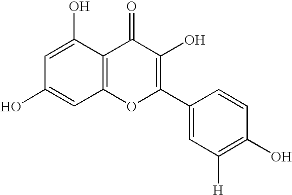 |
| Resveratrol | 5-[(E)-2-(4-hydroxyphenyl)-ethenyl]benzene-1,3-diol | 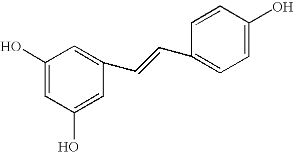 |
| Silibinin | 2,3-Dihydro-3-(4-hydroxy-3-methoxyphenyl)-2-(hydroxymethyl)-6-(3,5,7-trihydroxy-4-oxobenzopyran-2-yl)benzodioxin | 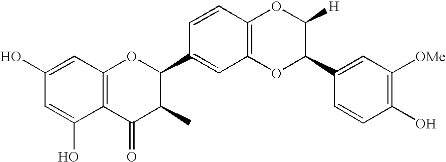 |

TABLE 4
Inhibition of UDPGT by bilirubin isomers, derivatives, and structural
analogues and flavonoids.

| Compound | | Apparent Ki |
|---|---|---|
| Name | Structure Formula | (μM) |
| Bilirubin IIIα | 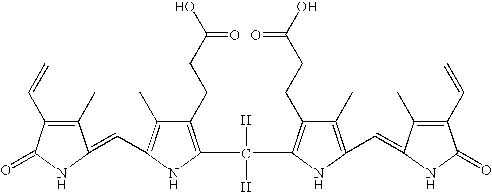 | 1.5 |
| Bilirubin XIIIα | 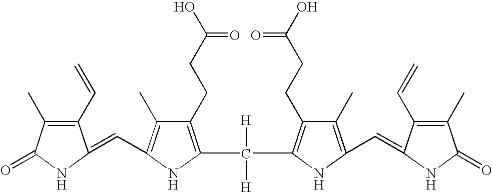 | 1.7 |
| Mesobilirubin IXα | 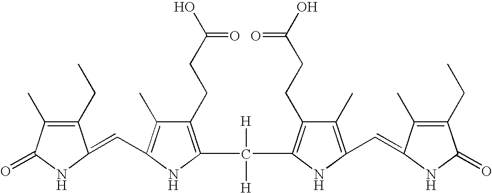 | 2.3 |
| Bilirubin IXβ | 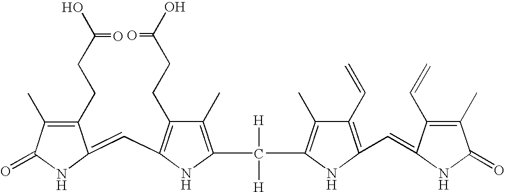 | 2.8 |
| DimethoxybilirubinIXα | 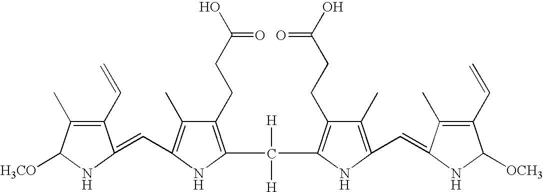 | 1.3 |
| Biliverdin (IXα) | 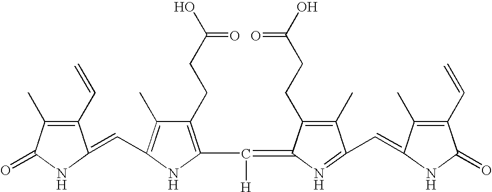 | 19.9 |
| Silybinin | See table 3 | 2.1 |
| Octyl gallate ester | See table 3 | 7.6 |
| Naringenin | See table 3 | 4.8 |
| Quercetin | See table 3 | 4.5 |

### References

Ames B. N., Shigenaga M. K. and Hagen T. M., Oxidants, antioxidants, and the degenerative diseases of aging, Proc. Natl. Acad. Sci. USA, 1993, 90:7915-7922.
Boiadjiev S. E. and Lightner D. A, A water soluble synthetic bilirubin with carboxyl groups replaced by sulfonyl moieties. Monatschefte fur Chemic, 2001, 132:1201-1212.
Boiadjiev S. E. and Lightner D. A., Novel benzoic acid congeners of bilirubin. Journal of organic Chemistry, 2003, 68:7591-7604.
Bosma P, Meer L, Bakker C., Hofman A. And Witterman C., UGTIA128 Allele and coronary heart disease: the Rotterdam study. Clinical Chem. 2003, 49:1180-1181.
Bratlid D., Billirubin toxicity: Pathophysiology and assessment of risk factors, New York State Journal of Medicine, 1991, 91:489-492.
Breimer L., Wannamethee G., Ebrathim S., and Shaper A., Serum bilirubin and risk of ischemic heart disease in middle-aged British men, Clinc. Chem., 1995, 41:1504-1508.
Chen Q., Wang T., Zhang Y. and Ma J., Large Scale efficient synthesis of 9-unsubstituted dipyrrinone. Synthetic Communications, 2002,32:1031-1040.
Deng Q., Detection of arsenic-trioxide in the Niuhuang Jiedu tablet. Zhong Yao Tong Bao. 1983 Jan;8(1):19-20. (in Chinese)
Dong S, Zhu Z, Zhang Y, Xu Z, Cheng J. Determination of 11 metal elements in niuhuang jiangya wan by atomic absorption spectrophotometer. Guang Pu Xue Yu Guang Pu Fen Xi. 1999;19(3):417-8. (in Chinese).
Djousse L., Rothman K., Cupples L., Levy D., and Ellison R., Serum albumin and risk of myocardial infarction and all-cause mortality in Framingham offspring study. Circulation, 2002, 106:2919-2924.
Endler G., Hamwi A., Plassmann R., and Wagner O., Is low serum bilirubin an independent risk factor for coronary artery disease in men but not in women? Clincal. Chem. 2003, 49:1201-1204.
Frei B., Stocker R., Ames B. N., Antioxidant defenses and lipid peroxidation in human blood plasma. Proc. Natl. Acad. Sci., 1988, 85:9748-9752.
Gullu H., Erdogan D., Tok D., Topeu S., Caliskan M., UIU.S. T., and Muderrisoglu, High serum bilirubin concentrations preserve coronary flow reserve and coronary mierovascular functions. Arterioscler Thromb Vasc Biol., 2005, 25:1-6.
Hansen T., Mechanisms of bilirubin toxicity: Clinical applications. Clin. Perinatol., 2002, 29:765-778.
Hopkins P., Wu L., Hunt S., James B., Vicent G., Williams R., Higher serum bilirubin is associated with descreased risk for early familial coronary disease. Artherosclerosis, Thrombosis, and Vascular Biology, 1996, 16:250-255.
Hu H., Zhang W., Wanf Z., Acute cardiovascular disease and serum bilirubin level. Journal of Clinical Cardiology, 2004, 20:180-181. (in Chinese)
Ishizaka N., Ishizaka Y., Takahashi E., Yamakado M. And Hashimoto H., High serum bilirubin level is inversely associated with the presence of carotid plaque. Stroke, 2001, 32:580-581.
Levinson, S., Relationship between bilirubin, apolipoproetin B, and coronary artery disease. Am. Clin. Lab. Sci., 1997, 27:185-192.
Lin Q., Xiong S., Xu S., Lin L., Lower serum bilirubin and oxidiatively modified low density lipoprotein are associated with coronary heart disease Journal of Clinical Cardiology, 2002, 18:204-206. (in Chinese).
Louise Y. and Meade T., Metal Complexes as Enzyme Inhibitors, Chem. Rev., 1999, 99:2711-2734.
Mayer M., 2000, Association of serum bilirubin concentration with risk of coronary artery disease. Clinical Chem., 2000, 46:1723-1727.
McPhee F., Caldera P., Bemis G., McDonaugh A., Kuntz I., and Craik C., Bile pigments as HIV protease inhibitors and their effects on HIV viral maturation and infectivity in vitro. Biochem, J., 1996, 320:681-686.
Neuzil J., Stocker R., Free and albumin-bound bilirubin are efficient co-antioxdiants for tocopherol, inhibiting plasma and low density lipoprotein lipid peroxidation. Hour. Biol. Chem., 1994, 269:16712-16719.
Olineseu R., Kummerow F., Greabu M., Crocnan D., and Voiculscu B., The levels of bilirubin may be related to an inflammatory condition in patients with coronary heart disease. Rom J. Intern Med., 1999; 37:239-249.
Pan I., Luo Y., Relationship between serum bilirubin and oxidatively modified low density lipoprotein. Chinese Journal of Clinical Medicine, 2005, 4:12-14. (in Chinese)
Papadakis J., Ganotakis E., Jagroop I., Mikhailidis D., and Winder A., Effect of hypertension and its treatment on lipid, lipoprotein, fibrinogen, and bilirubin levels in patients referred for dyslipidemia. Am J Hypertens., 1999, 12:673-681.
Peng D., Yu Y., Cheng Z., Hu Li., Gu H., Relationship between serum bilirubin and coronary disease. Journal of Clinical Cardiology, 2004, 20:18-21, (in Chinese)
Schwertner H. A., Serum bilirubin and liver function tests as risk predictors for coronary artery disease, 1995, U.S. Pat. No. 5,380,667.
Schwertner H., Association of smoking and low serum bilirubin antioxidant concentration. Atherosclerosis. 1998, 136:383-387.
Schwertner H. A., and Fisher J. R., Comparison of various lipid, lipoprotein, and bilirubin combinations as risk factors for predicting coronary artery disease. Atheroselerosis, 2000, 150:381-387.
Schwertner H. A., and Fisher J. R., Combined cholesterol and bilirubin tests as risk predictors for coronary artery disease, 2005, U.S. Pat. No. 6,869,802.
Schertner H. A., Jackson W., Tolan G., Association of low serum concentration of bilirubin with increased risk of coronary artery disease. Clinical Chem., 1994, 40:18-23.
Stocker, R., Antioxidant activities of bile pigments. Antioxidants & Redox Signaling, 2004, 6:841.
Stocker R., Glazer A. N., and Ames B. N., Antioxidant activity of alubumin-bound bilirubin, Proc. Natl. Acad. Sci., 1987, 84:5918-5922.
Sun, F., Lin Q., and Xu S. Relation between serum bilirubin and coronary heart disease. Journal of Clinical Diagnosis, 1999, 17:1-3.
Thyrann T. and Lightner D. A., Synthesis and properties of a lipid bilirubin analog. Tetrahedron., 1996, 52:447-460.
Vitek L., Jirsa M., Brodanova M., Kalab M., Marecek Z., and Kotal P., Gilbert syndrome and ischemic heart disease: a protective effect of elevated bilirubin levels. Artherosclerosis, 2002, 160:449-456.
Wang, L. M. Chronic arsenic poisoning caused by long-term taken Niuhuang Ninggong tablet. Zhongguo Zhong Xi Yi Jie He Za Zhi. 2005 March; 25(3):213. (in Chinese).
Wennberg R. P., Cellular basis of bilirubin toxicity, New York State Journal of Medicine, 1991, 91:493-496.
Zucker S. D., Horn P. S., and Sherman K. E., Serum bilirubin levels in the US population: gender effect and inverse correlation with colorectal cancer. Hepatology, 2004, 40:827-835.

I claim:

1. A composition for preventing, controlling or treating metabolic disorder, age-related disease, and acute inflammatory conditions comprising an effective amount of a compound or compounds selected from the group consisting of formulas (a)-(c):
linear tetrapyrroles 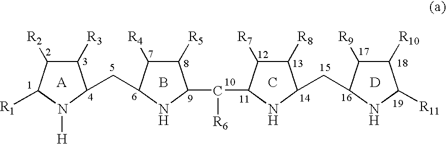
tripyrroles 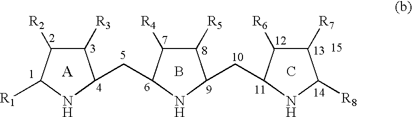
and dipyrroles 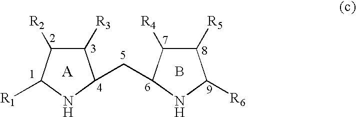
wherein A, B, C and D are pyrroles linked by methylene or methine groups, wherein the carbon position on the backbone are labeled numerically; and wherein the functional groups (R1, R2, . . . R10 and R11) are selected from the following:
hydrogen: —H;
oxygen: ═O;
hydroxyl: —OH;
straight chain or branched alkanes: —(CH₂)ₙCH₃;
straight chain or branched alkenes: —(CH₂)ₙCH═CH(Ch₂)ₘCH₃;
unsaturated straight chain or branched fatty acids: —(CH₂)ₙCH═CH(CH₂)ₘCO₂H;
straight or branched aliphatic fatty asters: —(CH₂)ₙCO₂CH₃, or —(CH₂)ₙCH═CH(CH₂)ₘCO₂CH₃, or —(CH₂)ₙCH₂CO₂(CH₂)ₘCH₃;
straight chain or branched aliphatic alcohols: —(CH₂)ₙCh₂OH, (CH₂)ₙCH═CH(CH₂)ₘCH₂OH;
alkoxyls: —O(CH₂)ₙCH₃;
alkylsulfonic acids: —(CH₂)ₙSO₃H, or —(CH₂)ₙCH═Ch(CH₂)ₘSO₃H;
alkylsulfate: —(CH₂)ₙOSO₃H, or —(CH₂)ₙCH═CH(CH₂)ₘOSO₃H; 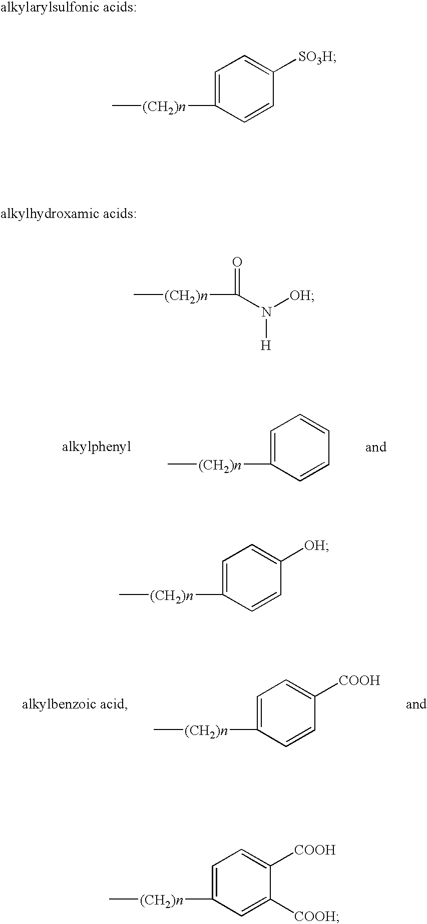
wherein the hydrocarbon chain length (n and m) ranges from 0 to 20.

2. The composition of claim 1, wherein the linear tetrapyrroles are selected from the group consisting of bilirubins, derivatives of bilirubins, and their structural analogues, which have formulas (i)-(x): 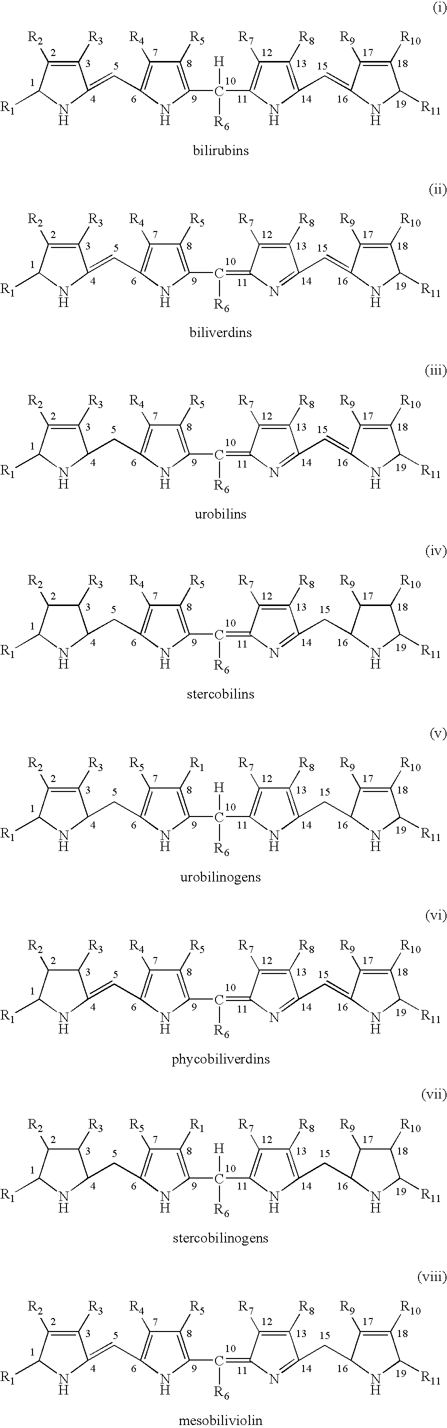 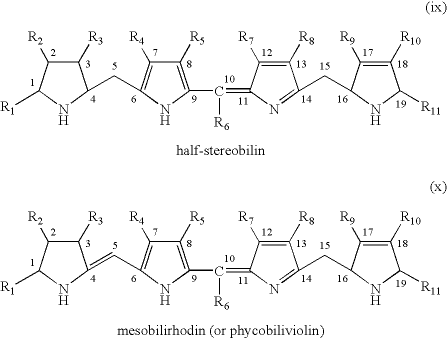
wherein the functional groups (R1, R2, . . . , and R11) are selected from the following:
hydrogen: —H;
oxygen: ═O;
hydroxyl: —OH;
straight chain or branched alkanes: —(CH₂)ₙCH₃;
straight chain or branched alkenes: —(Ch₂)ₙCH═CH(CH₂)ₘCH₃;
saturated straight chain or branched aliphatic fatty acids: —(CH₂)ₙCO₂H;
unsaturated straight chain or branched fatty acids: —(CH₂)ₙCH═CH(CH₂)ₘCO₂H;
straight or branched aliphatic fatty esters: —(CH₂)ₙCO₂CH₃, or —(CH₂)ₙCH═CH(CH₂)ₘCO₂CH₃, or —(CH₂)ₙCH₂CO₂(CH₂)ₘCH₃;
straight chain or branched aliphatic alcohols: —(CH₂)ₙCH₂OH, —(CH₂)ₙCH═CH(CH₂)ₘCH₂OH;
alkoxyls: —O(CH₂)ₙCH₃;
alkylsulfonic acids: —(CH₂)ₙSO₃H, or —(CH₂)ₙCH═CH(CH₂)ₘSO₃H;
alkylsulfate: —(CH₂)ₙOSO₃H, or —(CH₂)ₙCH═CH(CH₂)ₘOSO₃H; 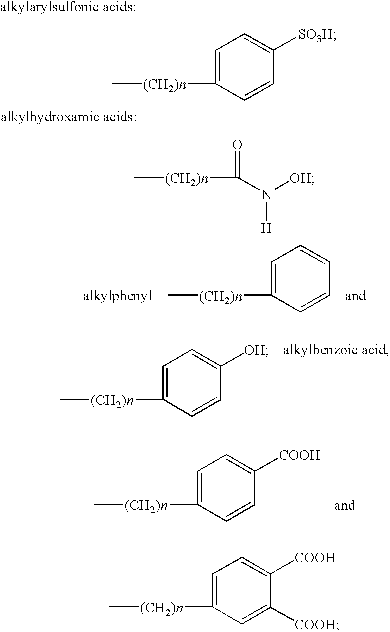
wherein the hydrocarbon chain length (n and M) ranges from 0 to 20.

3. The composition of claim 1, wherein the tripyrroles are selected from the group consisting of 1,14-dioxo tripyrroles having formulas (xi)-(xiii): 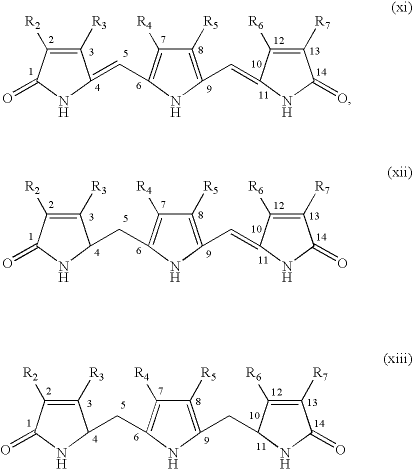
wherein the functional groups (R1, . . . , R₇, and R8) are selected from the following structures:
hydrogen: —H;
oxygen: ═O;
hydroxyl: —OH;
straight chain or branched alkanes: —(CH₂)ₙCH₃;
straight chain or branched alkenes: —(CH₂)ₙCH═CH(Ch₂)ₘCH₃;
saturated straight chain or branched aliphatic fatty acids: —(CH₂)ₙCO₂H;
unsaturated straight chain or branched fatty acids: —(CH₂)ₙCH═CH(CH₂)ₘCO₂H;
straight or branched aliphatic fatty esters: —(CH₂)ₙCO₂CH₃, or —(CH₂)ₙCH═CH(CH₂)ₘCO₂CH₃, or —(Ch₂)ₙCH₂CO₂(CH₂ₘCH₃;
straight chain or branched aliphatic alcohols: —(CH₂)ₙCH₂OH, —(CH₂)ₙCH═CH(CH₂)ₘCH₂OH;
alkoxyls: —O(CH₂)ₙCH₃;
alkylsulfonic acids: —(CH₂)ₙSO₃H, or —(CH₂)ₙCH═Ch(CH₂)ₘSO₃H;
alkylsulfate: —(CH₂)ₙOSO₃H, or —(CH₂)ₙCH═CH(CH₂)ₘOSO₃H; 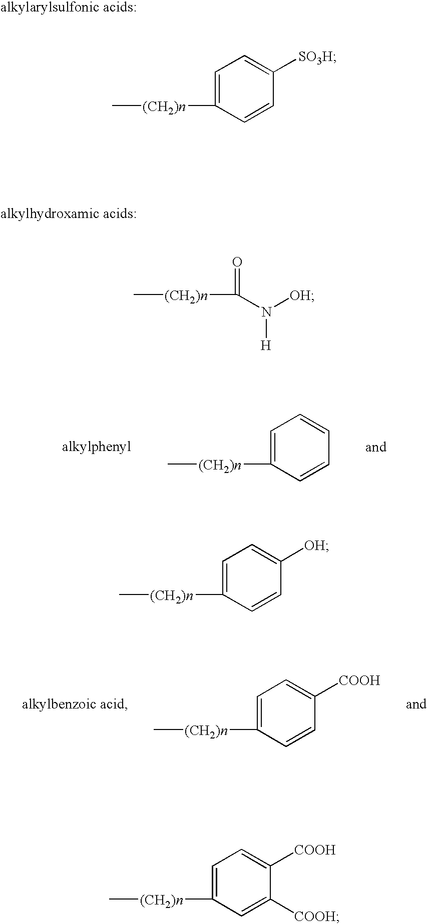
wherein the hydrocarbon chain length (n and m) ranges from 0 to 20.

4. The composition of claim 1, wherein the dipyrroles are selected from the group consisting of structure formulas (xiv) and (xv): 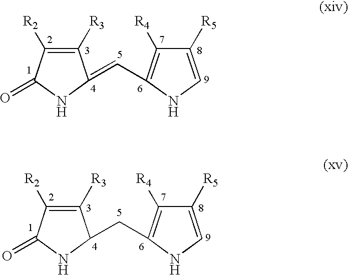
wherein the functional groups (R1, R2, R3, R4 and R5) are selected from the following structures:
hydrogen: —H;
oxygen: ═O;
hydroxyl: —OH;
straight chain or branched alkanes: —(CH₂)ₙCH₃;
straight chain or branched alkenes: —(CH₂)ₙCH═CH(CH₂)ₘCH₃;
saturated straight chain or branched aliphatic fatty acids: —(CH₂)ₙCO₂H;
unsaturated straight chain or branched fatty acids: —(Ch₂)ₙCH═CH(CH₂)ₘCO₂H;
straight or branched aliphatic fatty esters: —(CH₂)ₙCO₂CH₃, or —(CH₂)ₙCH═CH(CH₂)ₘCO₂CH₃, or —(CH₂)ₙCH₂CO₂(CH₂)ₘCH₃;
straight chain or branched aliphatic alcohols: –(CH₂)ₙCH₂OH, —(CH₂)ₙCH═CH(CH₂)ₘCH₂OH;
alkoxyls: —O(CH₂)ₙCH₃;
alkylsulfonic acids: —(CH₂)ₙSO₃H, or —(CH₂)ₙCH═CH(CH₂)ₘSO₃H;
alkylsulfate: —(CH₂)ₙOSO₃H, or —(CH₂)ₙCH═CH(CH₂)ₘOSO₃H; 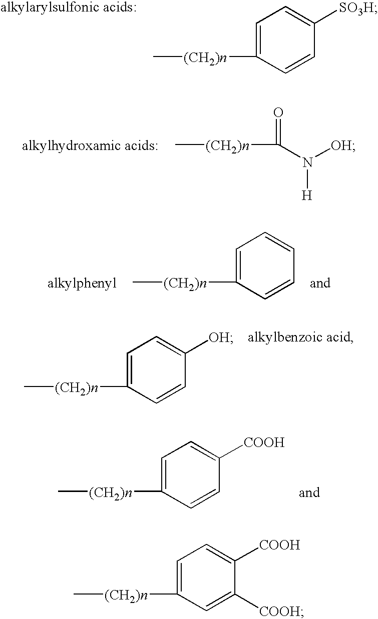
wherein the hydrocarbon chain length (n and m) ranges from 0 to 20.

5. The composition of claim 2, wherein the bilirubins and bilirubin analogues are selected from the group consisting of 1,19-bilindiones and having the following formula:

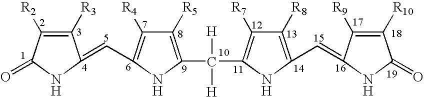

6. The composition of claim 2, wherein the biliverdins and biliverdin analogues are selected from the group consisting of 1,19-bilindiones and having the following structural formula

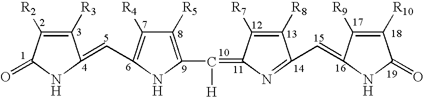

7. The composition of claim 2 wherein the bilirubin derivatives and analogues thereof are selected from the group consisting of 1,19-bilindiones having the following formulas:

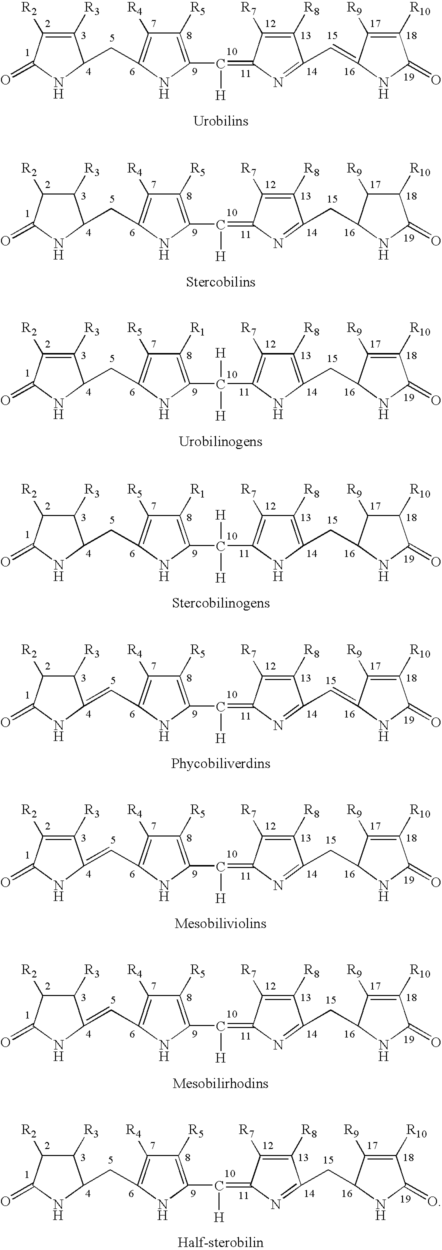

8. The composition of claim 5, wherein R5 and R7 are propionic acids, R2, R4, R8, and R9 are methyl R3 and R10 are ethyl or vinyl, and wherein the compound has the following formula: 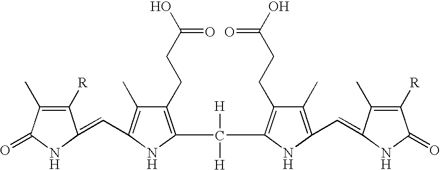
wherein R is vinyl or ethyl.

9. The composition of claim 8, wherein the bilirubin can be administered in the form of pre-drugs selected from the group consisting of heme, hematin, hemin, hemoglobin, myoglobin, or a protoporphyrrin compound and having the following formula

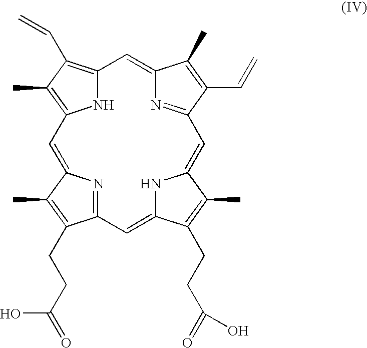

10. The composition of claim 1, wherein the bilirubins, derivatives of bilirubins, analogues of bilirubins and their derivatives, tripyrroles, dipyrroles, and heme analogues are formulated either with the compounds by themselves, or their complexes with metals consisting of sodium, potassium, calcium, magnesium, manganese, iron, zinc and copper, or their conjugates consisting of glucuronides, taurates, albumins, and amine acids, or the combination of two or more.

11. The composition of claim 1, wherein the therapeutic effects are enhanced by the addition of a bilirubin:uridine diphosphate glucuronosyltransferase (UDPGT) inhibitor selected from the group consisting of flavonoids, polyphenols, and phenyl aliphatic acids.

12. The composition of claim 11, wherein the flavonoids are selected from the group consisting of naringenin, apigenin, chrysin, hesperetin, tangeretin, quercetin, acacetin, nabilerin, and galangin.

13. The composition of claim 11, wherein the polyphenols are selected from the group consisting of kaempferol, resveratrol, silybinin, and alkyl esters of gallic acids.

14. The composition of claim 1, wherein the amount is effective to prevent, control or treat metabolic disorders manifested as a symptom or symptoms consisting of high levels of total blood cholesterol, triglyceride and LDL-cholesterol, high blood pressure, low levels of total serum bilirubin, overweight, and obesity.

15. The composition of claim 1, wherein the amount is effective to prevent, control or treat cardiovascular disease including coronary heart disease, atheroselerosis, and stroke.

16. The composition of claim 1, wherein the amount is effective to prevent, control or treat an autoimmune disease including rheumatoid arthritis, allergies, and organ rejection after transplantation, and acute inflammations including asthma and sunburn.

17. The composition of claim 1, wherein the amount is effective to prevent, control or treat cancers and Alzheimer's disease.

18. The composition of claim 1, wherein the compounds are formulated in a dosage form selected from the group consisting of pills, tablets, capsules, gels, chewing gum, syrup, injection, suppository, nasal spray, lotion, gel, spray, and patch.

19. The composition of claim 1, wherein the composition comprises an effective amount of the compound or compounds to administer a dosage in the range of from 0.001 to 100 mg/kg body weight, preferably in the range of from 0.01 to 50 mg/kg body weight, and most preferably in the range of from 0.05 to 10 mg/kg body weight.

20. A method of preventing, inhibiting or controlling high blood cholesterol, overweight, obesity, cardiovascular disease, rheumatoid arthritis, cancer, Alzheimer's disease, asthma, allergy, and sunburn comprising of administering to a patient in need thereof an effective amount of the composition of claim 1.

21. The method of claim 20, wherein the amount of the compound or compounds in the composition is effective to increase or maintain the total serum bilirubin level in the appropriate ranges for the patient.

22. The method of claim 20, Wherein the amount of the compound or compounds in the composition is effective to reduce or control the activity or amount or both of enzymes that control the absorption of fat and cholesterol from intestine.

23. The method of claim 20, wherein the amount of the compound or compounds in the composition is effective to reduce or control the activity or amount or both of tissue enzymes that initiate and/or control tissue inflammatory reactions.

24. The method of claim 20, wherein the amount of the compound or compounds in the composition is effective to reduce or control the activity or amount or both of acetylcholinesterase.

25. The method of claim 20, wherein the amount of the compound or compounds in the composition is effective to reduce or control the activity or amount or both of histone decaetylase, and induce cancer cell apoptosis.

26. The method of claim 20, wherein the amount of the compound or compounds in the composition is effective to modulate the levels of total serum bilirubin (TSB) and total cholesterol (TC) to maintain the ratio of TSB/TX in the range of from 100 to 400 when the concentration units are in mmol/L, or in the range of from 60 to 250 when the concentration units are in mg/dL.
